(19) United States
(12) Patent Application Publication
(10) Pub. No.: US 2009/0118141 A1
(43) Pub. Date: May 7, 2009

(54) METHODS OF OLIGOSACCHARIDE PROFILING FOR THE DETECTION OF OCULAR ROSACEA

(75) Inventors: Carlito B. Lebrilla, Davis, CA (US); Hyunjoo An, Davis, CA (US); Lenio S. Alvarenga, Sao Paulo (BR); Mark D. Mannis, Carmichael, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA (US)

(21) Appl. No.: 11/913,043

(22) PCT Filed: May 3, 2006
(86) PCT No.: PCT/US06/16838

§ 371 Date: Jul. 23, 2008

Related U.S. Application Data
(60) Provisional application No. 60/677,861, filed on May 4, 2005.

(51) Int. Cl. C40B 40/12; C07K 16/44; C07K 16/00; A61K 39/395; C07H 3/00; C12N 9/08; C12N 9/16

(52) U.S. Cl. 506/19; 530/387.5; 530/391.3; 424/137.1; 536/123.1; 435/192; 435/196

(57) ABSTRACT

The present invention provides methods for identifying oligosaccharides specific to an inflammatory or infectious disease, methods for diagnosing an inflammatory or infectious disease by detecting the presence or absence of such oligosaccharides, and methods for treating an inflammatory or infectious disease by administering antibodies directed to such oligosaccharides. The present invention also provides methods for diagnosing ocular rosacea by determining the presence or absence of specific oligosaccharide markers. In addition, the present invention provides markers for ocular rosacea comprising 0-linked oligosaccharides as well as kits for diagnosing or treating ocular rosacea.

### CROSS-REFERENCES TO RELATED APPLICATIONS

This application claims priority to U.S. Provisional Application No. 60/677,861, filed May 4, 2005, the disclosure of which is hereby incorporated by reference in its entirety for all purposes.

### STATEMENT AS TO RIGHTS TO INVENTIONS MADE UNDER FEDERALLY SPONSORED RESEARCH AND DEVELOPMENT

This invention was made with Government support under Grant (or Contract) No. RO1-GM049077, awarded by the National Institute of Health. The Government has certain rights in this invention.

### BACKGROUND OF THE INVENTION

Rosacea is a chronic cutaneous disorder characterized by persistent erythema, telangiectases, papules, and pustules primarily of the convexities of the central face, i.e., cheeks, chin, nose, and central forehead (Wilkin et al., J. Am. Acad. Dermatol., 46:584-587 (2002)). The disease frequently involves the eye and is manifested as ocular surface inflammation (Ghanem et al., Cornea, 22:230-233 (2003)). It is estimated that approximately 13 million Americans have rosacea (Zuber, Prim. Care, 27:309-318 (2000); Zuber, Hosp. Pract. (Off Ed), 32:188-189 (1997)). In a Swedish study, a prevalence of 10% was found Berg et al., Acta Derm. Venereol., 69:419-423 (1989)). Ocular rosacea is believed to affect more than 50% of patients with rosacea, although an incidence as low as 8% has recently been reported (Starr, Proc. R. Soc. Med., 62:9-11 (1969); Quarterman et al., Arch. Dermatol., 133:49-54 (1997); Michel et al., Ann. Dromatol. Venereol., 130:20-24 (2003)). The diagnosis of ocular rosacea is particularly challenging in a subgroup of patients that do not present with typical facial skin findings but have ocular signs and symptoms. Indeed, up to 90% of patients with ocular rosacea may have neither obvious roseatic skin changes nor a previous diagnosis of rosacea. Thus, ocular rosacea is a common disease with the potential for causing a range of ocular pathology and in its severest form may lead to blindness.

The diagnosis of ocular rosacea requires careful physical examination of the eye. An early study reported by Starr and McDonald in which the eyes of rosacea patients were examined found ocular complications in 58% with corneal involvement in 33%. More recently, Ghanem et al., supra, reported that ocular rosacea cannot always be diagnosed solely by ocular findings, even though 20% of patients develop eye manifestations before the emergence of skin findings. The authors suggested that a clinician's increased awareness of ocular findings, in particular lid disease-related symptoms, may aid in earlier clinical diagnosis and treatment.

Although an objective method for diagnosing ocular rosacea has eluded clinicians, techniques for the detection of ocular rosacea using biological markers could provide more a definitive diagnosis of the disease. However, analyses of human tears have been performed primarily by investigating the protein components of tear fluids. For example, proteomic analysis of human tears found that three human α-defensins were significantly up-regulated in their expression after ocular surface surgery and the levels decreased to approximately normal after 30 days, when healing was complete (Zhou et al., J. Proteome Res., 3:410-416 (2004)). The utility of such protein components as markers for diagnosing ocular rosacea is unknown.

Human tears are known to contain high molecular weight glycoproteins such as mucins as major components. Mucin glycoproteins are highly glycosylated and can be about 80% glycans in composition (Prydal et al., Eye, 7:472-475 (1993)). Specifically, oligosaccharides, primarily O-linked or mucin-type oligosaccharides, make up a large fraction of the mass of mucins (Van den Steen et al., Crit. Rev. Biochem. Mol. Biol., 33:151-208 (1998)). In addition, oligosaccharides such as those found on mucins are known to be sensitive to the biochemical environment. Although several attempts have been made to identify the mucins expressed in tear fluid (Ellingham et al., Glycobiology, 9:1181-1189 (1999); Pflugfelder et al., Invest. Opthalmol. Vis. Sci., 41:1316-1326 (2002); McKenzie et al., Invest. Opthalmol. Vis. Sci., 41:703-708 (2000)), the identification, characterization, and utility of oligosaccharides released from mucins or other glycoproteins in tear fluid is unknown.

As such, there is a need in the art for a glycomic approach to identify suitable oligosaccharide markers for diagnosing inflammatory or infectious diseases such as ocular rosacea. The present invention satisfies this and other needs.

### BRIEF SUMMARY OF THE INVENTION

The present invention provides methods for identifying oligosaccharides specific to an inflammatory or infectious disease, methods for diagnosing an inflammatory or infectious disease by detecting the presence or absence of such oligosaccharides, and methods for treating an inflammatory or infectious disease by administering antibodies directed to such oligosaccharides. The present invention also provides methods for diagnosing ocular rosacea by determining the presence or absence of specific oligosaccharide markers. In addition, the present invention provides markers for ocular rosacea comprising O-linked oligosaccharides as well as kits for diagnosing or treating ocular rosacea.

As such, in one aspect, the present invention provides a method for identifying an oligosaccharide specific to an inflammatory or infectious disease, the method comprising:
(a) selectively releasing the oligosaccharides from a test sample, wherein the test sample is a sample from an individual having the inflammatory or infectious disease;
(b) obtaining a mass spectrum of the oligosaccharides from the test sample using matrix-assisted laser desorption ionization (MALDI)-Fourier transform mass spectrometry (FTMS); and
(c) comparing the mass spectrum from the test sample to the mass spectrum from a control sample,
   wherein the oligosaccharide specific to the inflammatory or infectious disease is identified by the presence of a unique oligosaccharide in the mass spectrum from the test sample.

In another aspect, the present invention provides a method for diagnosing an inflammatory or infectious disease in an individual, the method comprising:
detecting the presence or absence of a unique oligosaccharide in a sample from the individual, wherein the presence of the unique oligosaccharide indicates that the individual has the inflammatory or infectious disease.

In yet another aspect, the present invention provides a method for treating an inflammatory or infectious disease in an individual in need thereof, the method comprising:
administering to the individual a therapeutically effective amount of an antibody that binds specifically to a unique oligosaccharide.

In still yet another aspect, the present invention provides a method for diagnosing ocular rosacea in an individual, the method comprising:
(a) obtaining a mass spectrum of the oligosaccharides from a sample from the individual using MALDI-FTMS, wherein the oligosaccharides have been selectively released from the sample; and
(b) comparing the mass spectrum from the sample to the mass spectrum from a control sample,
   wherein the presence of a higher abundance of anionic oligosaccharides in the sample indicates that the individual has ocular rosacea.

In a related aspect, the present invention provides a method for diagnosing ocular rosacea in an individual, the method comprising:
(a) obtaining a mass spectrum of the oligosaccharides from a sample from the individual using MALDI-FTMS, wherein the oligosaccharides have been selectively released from the sample; and
(b) comparing the sum of the absolute intensities of anionic oligosaccharides in the mass spectrum from the sample to the sum of the absolute intensities of anionic oligosaccharides in the mass spectrum from a control sample,
   wherein the presence of a higher value for the sum of the absolute intensities of anionic oligosaccharides in the sample indicates that the individual has ocular rosacea.

In a further aspect, the present invention provides a method for diagnosing ocular rosacea in an individual, the method comprising:
(a) obtaining a mass spectrum of the oligosaccharides from a sample from the individual using MALDI-FTMS, wherein the oligosaccharides have been selectively released from the sample; and
(b) determining the presence or absence of a marker for ocular rosacea selected from the group consisting of a sulfated oligosaccharide, a NeuAc-containing oligosaccharide, a NeuGc-containing oligosaccharide, a HexA-containing oligosaccharide, a Hex-containing oligosaccharide, and combinations thereof in the mass spectrum,
   wherein the presence of the marker indicates that the individual has ocular rosacea.

In another aspect, the present invention provides an O-liked oligosaccharide having a composition selected from the group consisting of [HexNAc]₂[SO₃H], [HexNAc]₂[Hex]₁[SO₃H], [HexNAc]₂[Hex]₂[SO₃H], [HexNAc]₂[Hex]₃[SO₃H], [HexNAc]₂[HexA]₁[SO₃H], [HexNAc]₂[HexA]₁[Hex]₁[SO₃H], [HexNAc]₂[HexA]₁[Hex]₂[SO₃H], [HexNAc]₃[NeuAc]₂, [HexNAc]₃[NeuAc]₂[Hex]₁, [HexNAc]₃[NeuAc]₂[Hex]₂, [HexNAc]₃[NeuAc]₂[Hex]₃, [HexNAc]₁[NeuGc]₃[Hex]₂, [HexNAc]₁[NeuGc]₃[Hex]₃, [HexNAc]₁[NeuGc]₃[Hex]₄, [HexNAc]₁[NeuGc]₃[Hex]₅, m/z 607+[HexA]₁, m/z 607+[HexA]₂, m/z 607+[HexA]₃, m/z 607+[HexA]₄, m/z 607+[HexA]₅, m/z 591+[HexA]₁, m/z 591+[HexA]₂, m/z 591+[HexA]₃, m/z 997+[HexA]₁, m/z 997+[HexA]₂, m/z 576+[HexA]₁, m/z 576+[HexA]₂, M/Z 576+[HexA]₃, m/z 1107+[Hex]₁, m/z 1107+[Hex]₂, m/z 383+[Hex]₁, m/z 383+[Hex]₂, m/z 789+[Hex]₁, m/z 789+[Hex]₂, m/z 1194+[Hex]₁, m/z 1194+[Hex]₂, m/z 1049+[Hex]₁, m/z 1049+[Hex]₂, and combinations thereof.

In yet another aspect, the present invention provides a method for treating ocular rosacea in an individual in need thereof, the method comprising:
administering to the individual a therapeutically effective amount of an antibody that binds specifically to an O-linked oligosaccharide.

In still yet another aspect, the present invention provides a kit for diagnosing ocular rosacea in an individual, the kit comprising:
(a) an array comprising a plurality of O-linked oligosaccharides;
(b) a plurality of antibodies that binds specifically to the plurality of O-linked oligosaccharides on the array; and
(c) directions for use of the array and the plurality of antibodies with a sample from the individual.

In a further aspect, the present invention provides a kit for treating ocular rosacea in an individual in need thereof, the kit comprising:
(a) an antibody that binds specifically to an O-linked oligosaccharide; and
(b) directions for use of the antibody.

Other objects, features, and advantages of the present invention will be apparent to one of skill in the art from the following detailed description and figures.

### BRIEF DESCRIPTION OF THE DRAWINGS

FIG. 1 shows the MALDI-FTMS spectra of control tear fluid samples (type A) in the negative mode. FIGS. 1A-C represent the mass spectrum obtained from 3 different individuals who did not have ocular rosacea.

FIG. 2 shows the MALDI-FTMS spectra of control tear fluid samples (type B) in the negative mode. FIGS. 2A-B represent the mass spectrum obtained from 2 different individuals who did not have ocular rosacea.

FIG. 3 shows representative MALDI-FTMS spectra of test tear fluid samples in the negative mode having predominantly (A) sialylated oligosaccharides; (B) sulfated oligosaccharides; or (C) hexuronic acid oligosaccharides.

FIG. 4 shows the sum of the absolute intensities of the anionic oligosaccharides found in the control and test (patient) samples, arranged in increasing order.

FIG. 5 shows box plots of the sum of the absolute intensities of the anionic oligosaccharides for the control and test (ocular rosacea) groups.

FIG. 6 shows the receiver operating characteristic (ROC) curve of using the sum of the absolute intensities of the anionic oligosaccharides as the marker. The area under the ROC curve is 0.9911 with a 95% confidence interval (0.9712, 1.000).

### DETAILED DESCRIPTION OF THE INVENTION

### I. General Overview

The present invention provides methods for identifying oligosaccharides specific to an inflammatory or infectious disease, methods for diagnosing an inflammatory or infectious disease by detecting the presence or absence of such oligosaccharides, and methods for treating an inflammatory or infectious disease by administering antibodies directed to such oligosaccharides. The present invention also provides methods for diagnosing ocular rosacea by determining the presence or absence of specific oligosaccharide markers. In addition, the present invention provides markers for ocular rosacea comprising O-linked oligosaccharides as well as kits for diagnosing or treating ocular rosacea.

The present invention is based on the discovery that O-linked oligosaccharides released from tear fluid are indicators of ocular rosacea. Although there have been a number of studies on the proteins, glycoproteins, and mucin glycoproteins in tear fluid, nothing was known about the composition of oligosaccharides in tear fluid. As such, this is the first study to provide a comprehensive characterization of O-linked oligosaccharides that are specific to ocular rosacea. In particular, Example 1 illustrates that samples from patients with ocular rosacea have a higher abundance of anionic oligosaccharides compared to control samples. Further, Example 1 shows that specific sulfated oligosaccharides, NeuAc-containing oligosaccharides, NeuGc-containing oligosaccharides, HexA-containing oligosaccharides, and/or Hex-containing oligosaccharides are found only in samples from patients with ocular rosacea. Such oligosaccharides are particularly useful as markers for detecting ocular rosacea in an individual.

### II. Definitions

As used herein, the following terms have the meanings ascribed to them unless specified otherwise.

The term “inflammatory disease” refers to a disease or disorder characterized or caused by inflammation. “Inflammation” refers to a local response to cellular injury that is marked by capillary dilatation, leukocytic infiltration, redness, heat, and/or pain that serves as a mechanism initiating the elimination of noxious agents and of damaged tissue. The site of inflammation includes the eye, the skin, the lungs, the pleura, a tendon, a lymph node or gland, the brain, the spinal cord, the intestinal tract, nasal and pharyngeal mucous membranes, a muscle, bone or bony tissue, a joint, the urinary bladder, the genitals, the uvula, the vagina, the cervix of the uterus, the canthus, the vertebrae, the rectum, the anus, a bursa, a follicle, and the like. Such inflammatory diseases include, but are not limited to, rosacea (e.g., ocular rosacea), inflammatory bowel disease, rheumatoid diseases (e.g., rheumatoid arthritis), other arthritic diseases, fibrositis, pelvic inflammatory disease, acne, psoriasis, actinomycosis, dysentery, biliary cirrhosis, Lyme disease, heat rash, Stevens-Johnson syndrome, mumps, pemphigus vulgaris, systemic lupus erythematosus, autoimmune hepatitis, and blastomycosis. Preferably, the inflammatory disease is rosacea or ocular rosacea. As such, in certain aspects, the present invention provides methods for identifying oligosaccharides specific to an inflammatory disease, methods for diagnosing an inflammatory disease by detecting the presence or absence of such oligosaccharides, and methods for treating an inflammatory disease by administering antibodies directed to such oligosaccharides.

The term “rosacea” refers to a chronic cutaneous disorder characterized by persistent erythema, telangiectases, papules, and pustules primarily of the convexities of the central face (i.e., cheeks, chin, nose, and central forehead). Rosacea is generally divided into four subtypes: ocular rosacea, erythematotelangiectatic rosacea, papulopustular rosacea, and phymatous rosacea. Many patients experience characteristics of more than one subtype at the same time. “Ocular rosacea” refers to a subtype of rosacea that is typically manifested as ocular surface inflammation. Patients with ocular rosacea commonly experience irritation of the lids and the eye. Other symptoms include, for example, styes, blepharitis, episcleritis, and chronically red eyes. Ocular rosacea may also affect the cornea, causing neovascularization, infections, and ulcers.

One skilled in the art will appreciate that rosacea can also be classified as an infectious disease. For example, rosacea is associated with infections from the infestation of the mite Demodex folliculorum (see, e.g., Powell, Cutis, 74 (3 Suppl):9-12, 32-34 (2004)). In addition, rosacea is associated with infections from bacteria such as Helicobacter pylori or Staphylococcus aureus (see, e.g., Zandi et al., East Mediterr. Health J, 9:167-171 (2003); Dahl et al., J. Am. Acad. Dermatol., 50:266-272 (2004)). As such, in certain aspects, the present invention provides methods for identifying oligosaccharides specific to an infectious disease, methods for diagnosing an infectious disease by detecting the presence or absence of such oligosaccharides, and methods for treating an infectious disease by administering antibodies directed to such oligosaccharides.

The term “infectious disease” refers to a disease that is caused by or capable of being transmitted by infection. Typically, the infectious disease is caused by a pathogenic microorganism or agent. Such infectious diseases include, without limitation, rosacea (e.g., ocular rosacea), acquired immune deficiency syndrome (AIDS), anthrax, arboviral neuroinvasive and non-neuroinvasive diseases (e.g., West Nile virus), botulism, brucellosis, chancroid, gential infections (e.g., Chlamydia trachomatis), cholera, coccidioidomycosis, cryptosporidiosis, cyclosporiasis, diphtheria, ehrlichiosis, enterohemorrhagic Escherichia coli, giardiasis, gonorrhea, Haemophilus influenzae, leprosy, hantavirus pulmonary syndrome, hepatitis, HIV infection, legionellosis, listeriosis, Lyme disease, malaria, measles, meningococcal disease, mumps, pertussis, plague, polio, psittacosis, rabies, Rocky Mountain spotted fever, rubella, salmonellosis, severe acute respiratory syndrome (SARS), shigellosis, smallpox, streptococcal disease, syphilis, tetanus, trichinosis, tuberculosis, tularemia, typhoid fever, varicella, and yellow fever. One skilled in the art will understand that certain inflammatory diseases such as rosacea and Lyme disease can also be classified as infectious diseases.

The term “sample” refers to any biological specimen obtained from an individual. Suitable samples for use in the present invention include, without limitation, whole blood, plasma, serum, saliva, urine, stool, tears, and any other bodily fluid or tissue. In a preferred embodiment, the sample is a tear fluid sample. The term also encompasses any specimen obtained from a cell line including, without limitation, conditioned medium and a cellular extract such as a membrane extract, a cytosolic extract, a nuclear extract, etc. One skilled in the art will understand that samples such as tear fluid or conditioned medium can be diluted prior to analysis.

The term “individual” refers to any animal, preferably a mammal, and more preferably a human.

The term “individual having an inflammatory or infectious disease” refers to an individual that has exhibited one or more symptoms associated with an inflammatory or infectious disease at the time the test sample is obtained, or has previously been diagnosed as having an inflammatory or infectious disease at the time the test sample is obtained.

The term “individual not having an inflammatory or infectious disease” refers to an individual that has not exhibited any symptoms associated with an inflammatory or infectious disease at the time the control sample is obtained, or is in remission from the symptoms associated with an inflammatory or infectious disease at the time the control sample is obtained, or has not exhibited any recurrence of a previously diagnosed inflammatory or infectious disease at the time the control sample is obtained. As such, an individual not having an inflammatory or infectious disease need not be distinct from an individual having an inflammatory or infectious disease. For example, an individual can provide samples at different times, e.g., once prior to having an inflammatory or infectious disease (control sample) and once while having the inflammatory or infectious disease (test sample), or a control sample can be obtained from an individual in remission or following therapy and compared to a test sample obtained from the same individual at an earlier time, e.g., when the individual had the inflammatory or infectious disease.

The term “marker” refers to any molecule that is detectable in a biological sample and indicative of a disease, disorder, or a susceptibility to a disease or disorder. Preferably, the marker is a glycan or sugar molecule (e.g., a monosaccharide, oligosaccharide, or polysaccharide). In preferred embodiments of the present invention, the marker is a unique oligosaccharide species that is specific to an inflammatory or infectious disease such as ocular rosacea. For example, the unique oligosaccharide species can be an O-linked oligosaccharide, an N-linked oligosaccharide, or combinations thereof.

The term “marker for ocular rosacea” refers to a marker that is indicative of ocular rosacea in an individual. The term encompasses a molecule that is present in a tear fluid sample, but whose level is modulated in tear fluid from an individual with ocular rosacea compared to normal tear fluid (i.e., tear fluid from an individual not having ocular rosacea). More particularly, the term includes, without limitation: (1) a molecule that is specifically present in tear fluid from an individual with ocular rosacea but not present in normal tear fluid; (2) a molecule whose level is increased in tear fluid from an individual with ocular rosacea compared to normal tear fluid; (3) a molecule that is specifically present in normal tear fluid but not present in tear fluid from an individual with ocular rosacea; and (4) a molecule whose level is reduced in tear fluid from an individual with ocular rosacea compared to normal tear fluid. In preferred embodiments of the present invention, the marker for ocular rosacea is an O-linked oligosaccharide. For example, the O-linked oligosaccharide can be a sulfated oligosaccharide, a NeuAc-containing oligosaccharide, a NeuGc-containing oligosaccharide, a HexA-containing oligosaccharide, a Hex-containing oligosaccharide, or combinations thereof. In other embodiments, the marker for ocular rosacea is an N-linked oligosaccharide.

The term “m/z” refers to the mass-to-charge ratio obtained by dividing the mass of an ion by its charge number.

The term “exoglycosidase” refers to an enzyme that selectively hydrolyzes a linkage between two monosaccharides. Preferably, the exoglycosidase selectively hydrolyzes a linkage between two monosaccharides present in an oligosaccharide (e.g., O-linked or N-linked oligosaccharide). Suitable exoglycosidases include, without limitation, fucosidases such as α1,2-fucosidase, α1-3,4-fucosidase, and α1,6-fucosidase; galactosidases such as α1-3,6-galactosidase, β1,3-galactosidase, β1-3,6-galactosidase, and β1,4-galactosidase; hexosaminidases such as α-N-acetyl-galactosaminidase and β1-2,3,4,6-N-acetyl-glucosaminidase; hexosidases such as β-glycosidase; mannosidases such as α1-2,3-mannosidase, α1,6-mannosidase, and β1,4-mannosidase; neuraminidases such as β2-3,6-neuraminidase, β2-3,6,8-neuraminidase, α2-3,6,8,9-neuraminidase, and α2,3-neuraminidase; xylosidases such as β1,2-xylosidase; and combinations thereof.

As used herein, the term “array” refers to an array of distinct nucleic acids, peptides, polypeptides, proteins, or oligosaccharides immobilized on a solid support or substrate such as paper, a membrane (e.g., nylon), a filter, a chip, a pin, a bead, glass (e.g., a glass slide), or any other suitable solid support. Preferably, the array comprises a plurality of different oligosaccharide species (e.g., O-linked oligosaccharides, N-linked oligosaccharides, etc.) that are coupled to the substrate surface in different known locations.

The term “therapeutically effective amount” refers to the amount of an antibody or other therapeutic agent (e.g., ions, small organic molecules, peptides, proteins, polypeptides, oligosaccharides, etc.) that is capable of achieving a therapeutic effect in a subject in need thereof. For example, a therapeutically effective amount of an antibody can be the amount that is capable of preventing or relieving one or more symptoms associated with an inflammatory or infectious disease such as ocular rosacea. Preferably, the antibody binds to a marker (e.g., unique oligosaccharide species) that is specific to ocular rosacea.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. One skilled in the art will know of additional methods for administering a therapeutically effective amount of an antibody or other therapeutic agent for preventing or relieving one or more symptoms associated with an inflammatory or infectious disease such as ocular rosacea.

### III. Description of the Embodiments

In one aspect, the present invention provides a method for identifying an oligosaccharide specific to an inflammatory or infectious disease, the method comprising:
(a) selectively releasing the oligosaccharides from a test sample, wherein the test sample is a sample from an individual having the inflammatory or infectious disease;
(b) obtaining a mass spectrum of the oligosaccharides from the test sample using matrix-assisted laser desorption ionization (MALDI)-Fourier transform mass spectrometry (FTMS); and
(c) comparing the mass spectrum from the test sample to the mass spectrum from a control sample,
   wherein the oligosaccharide specific to the inflammatory or infectious disease is identified by the presence of a unique oligosaccharide in the mass spectrum from the test sample.

The term “selectively releasing” refers to methods known to one skilled in the art for releasing primarily O-linked oligosaccharides, N-linked oligosaccharides, or a combination thereof from a sample. For example, methods using sodium borohydride (NaBH₄) as described in Morelle et al., Eur. J. Biochemistry, 252:253-260 (1998) and methods using hydrazine as described in Patel et al., Biochemistry, 32:679-693 (1993) are suitable for selectively releasing the O-linked oligosaccharides from a sample. Methods using PNGase F as described in Fan et al., J. Biol. Chem., 272:27058-27064 (1997); Tarentino et al., Methods Enzymol., 230:44-57 (1994); and Trimble et al., J. Biol. Chem., 266:1646-1651 (1991) are suitable for selectively releasing the N-linked oligosaccharides from a sample. With respect to O-linked oligosaccharides, the term encompasses the release of at least about 70%, preferably at least about 80%, more preferably at least about 90%, and most preferably at least about 95% of the O-linked oligosaccharides from a sample and the release of less than about 30%, preferably less than about 20%, more preferably less than about 10%, and most preferably less than about 5% of all other species such as non-O-linked oligosaccharides (e.g., N-linked oligosaccharides), proteins, peptides, and nucleic acids from the sample. Similarly, with respect to N-linked oligosaccharides, the term encompasses the release of at least about 70%, preferably at least about 80%, more preferably at least about 90%, and most preferably at least about 95% of the N-linked oligosaccharides from a sample and the release of less than about 30%, preferably less than about 20%, more preferably less than about 10%, and most preferably less than about 5% of all other species such as non-N-linked oligosaccharides (e.g., O-linked oligosaccharides), proteins, peptides, and nucleic acids from the sample.

The term “sample” refers to any biological specimen obtained from an individual. Suitable samples for use in the present invention include, without limitation, whole blood, plasma, serum, saliva, urine, stool, tears, and any other bodily fluid or tissue. Preferably, the sample (i.e., test sample or control sample) is tear fluid. The sample can be obtained from the individual by any method known to one skilled in the art. For example, a tear fluid sample can be collected using microcapillary tubes as described in Example 1 below or using polyester rods as described in Jones et al., Cornea, 16:450-458 (1997). Further, a whole blood sample can be obtained by drawing blood from an individual. Alternatively, a whole blood sample can be obtained from a blood bank or from a physician's office. The term also encompasses any specimen obtained from a cell line including, without limitation, conditioned medium and a cellular extract such as a membrane extract, a cytosolic extract, a nuclear extract, etc.

As used herein, the term “test sample” refers to a sample from an individual having an inflammatory or infectious disease. Non-limiting examples of inflammatory and infectious diseases are described above. Preferably, the individual has ocular rosacea. An “individual having an inflammatory or infectious disease” refers to an individual that has exhibited one or more symptoms associated with an inflammatory or infectious disease at the time the test sample is obtained, or has previously been diagnosed as having an inflammatory or infectious disease at the time the test sample is obtained.

As used herein, the term “control sample” refers to a sample from an individual not having the inflammatory or infectious disease. An “individual not having an inflammatory or infectious disease” refers to an individual that has not exhibited any symptoms associated with an inflammatory or infectious disease at the time the control sample is obtained, or is in remission from the symptoms associated with an inflammatory or infectious disease at the time the control sample is obtained, or has not exhibited any recurrence of a previously diagnosed inflammatory or infectious disease at the time the control sample is obtained.

One skilled in the art will appreciate that an individual not having an inflammatory or infectious disease need not be distinct from an individual having an inflammatory or infectious disease. For example, an individual can provide samples at different times, e.g., once prior to having an inflammatory or infectious disease (control sample) and once while having the inflammatory or infectious disease (test sample), or a control sample can be obtained from an individual in remission or following therapy and compared to a test sample obtained from the same individual at an earlier time, e.g., when the individual had the inflammatory or infectious disease.

In the methods of the present invention, the mass spectrum of the oligosaccharides (e.g., O-linked and/or N-linked oligosaccharides) from the test sample and the control sample are obtained using mass spectrometry techniques such as matrix-assisted laser desorption ionization (MALDI)-Fourier transform mass spectrometry (FTMS). MALDI is particularly advantageous because it facilitates desorption and ionization of biomolecules such as carbohydrates, nucleic acids, and proteins without their fragmentation. For example, Hillenkamp et al., Anal. Chem., 63:1193A-1203A (1991); Karas et al., Anal. Chem., 60:2299-2301 (1988); and Stahl et al., Anal. Biochem., 223:218-226 (1994) describe the application of MALDI to biomolecules. FTMS is a very high resolution mass spectrometry technique based on the magnetic trapping of ions and the excitation/detection of their cyclotron frequencies. As a result, FTMS can provide accurate mass measurements, i.e., <10 ppm routinely, <5 ppm with internal calibration. The application of MALDI-FTMS to the identification of oligosaccharide species in a sample is described, for example, in Tseng et al., Anal. Biochem., 250:18-28 (1997) and Tseng et al., Anal. Chem., 71:3747-3754 (1999).

As used herein, the term “obtaining a mass spectrum of the oligosaccharides” refers to obtaining an oligosaccharide profile for a sample (i.e., test sample or control sample) that contains either all of the O-linked and/or N-linked oligosaccharide species released from the sample or a fraction thereof. For example, when O-linked oligosaccharides are selectively released and purified by a technique such as solid phase extraction on a porous graphitized carbon (PGC) column, depending on the percentage (e.g., 10%, 20%, 40%) and/or type of solvent (e.g., acetonitrile) used, only a fraction of the released O-linked oligosaccharide species is eluted from the column. A mass spectrum obtained on the fraction can contain, for example, less than 1%, at least 1%, at least 5%, at least 10%, or at least 20% of the released O-linked oligosaccharide species. One skilled in the art will appreciate that the fraction of the released O-linked and/or N-linked oligosaccharide species present in the mass spectrum can also depend on whether the mass spectrum is obtained in the positive mode or the negative mode.

As used herein, the term “comparing the mass spectrum from a test sample to the mass spectrum from a control sample” refers to comparing or aligning the mass spectrum profile from a test sample to the mass spectrum profile from a control sample and determining any similarities and/or differences between the two mass spectrum profiles. One skilled in the art will appreciate that the mass spectrum profile can be a set of peaks, a set of m/z ratios, a set of rudimentary compositions, or in any other format suitable for comparing the oligosaccharides from a test sample to a control sample. The control mass spectrum profile can be obtained at the same time as the test mass spectrum profile or, alternatively, the control mass spectrum profile can be obtained previously and stored, for example, in a database. One skilled in the art will also appreciate that the two-mass spectrum profiles can be compared by a computer, e.g., using datasets. In certain instances, a computer software program compares and/or interprets the two mass spectrum profiles, e.g., using peak matching techniques. In certain other instances, an internet application compares and/or interprets the two mass spectrum profiles.

As used herein, the term “oligosaccharide specific to an inflammatory or infectious disease is identified by the presence of a unique oligosaccharide in the mass spectrum from the test sample” refers to the identification of those O-linked and/or N-linked oligosaccharides whose levels have modulated in the test sample compared to their levels in the control sample. More particularly, the term encompasses any O-linked or N-linked oligosaccharide species that is present in the test sample but is absent from the control sample, any O-linked or N-linked oligosaccharide species whose level is enhanced in the test sample compared to the control sample, any O-linked or N-linked oligosaccharide species whose level is reduced in the test sample compared to the control sample, and any O-linked or N-linked oligosaccharide species that is present in the control sample but is absent from the test sample.

In one embodiment, the method further comprises the step of subjecting the unique oligosaccharide or oligosaccharides to infrared multiphoton dissociation (IRMPD). The use of IRMPD is advantageous over techniques such as collision-induce dissociation (CID) because IRMPD does not limit the m/z range of observed product ions, eliminates the need for matching resonant frequencies, and allows more sequence information to be obtained, typically down to the last residue. As a result, the complete sequencing of even large oligosaccharides can be performed in a single experiment. In another embodiment, the method further comprises the step of digesting the unique oligosaccharides with an exoglycosidase. Preferably, the exoglycosidase selectively hydrolyzes a linkage between two monosaccharides present in the oligosaccharide. Suitable exoglycosidases for use in the present invention include any of the fucosidases, galactosidases, hexosaminidases, hexosidases, mannosidases, neuraminidases, and xylosidases described above or any other exoglycosidase known to one skilled in the art.

In another embodiment, the unique oligosaccharide is an O-linked oligosaccharide selected from the group consisting of an N-acetylneuraminic acid NeuAc)-containing oligosaccharide, an N-glycolylneuraminic acid (NeuGc)-containing oligosaccharide, a sulfated oligosaccharide, a hexose (Hex)-containing oligosaccharide, and a combination thereof.

In yet another embodiment, the present invention provides an antibody that binds specifically to a unique oligosaccharide identified by the above-described method. “Antibody” refers to a polypeptide comprising a framework region from an immunoglobulin gene or fragments thereof that specifically binds and recognizes an antigen such as a unique oligosaccharide of the present invention. An exemplary immunoglobulin (antibody) structural unit comprises a tetramer. Each tetramer is composed of two identical pairs of polypeptide chains, each pair having one “light” (about 25 kDa) and one “heavy” chain (about 50-70 kDa). Light chains are classified as either kappa or lambda. Heavy chains are classified as gamma, mu, alpha, delta, or epsilon, which in turn define the immunoglobulin classes, IgG, IgM, IgA, IgD, and IgE, respectively. The N-terminus of each chain defines a variable region of about 100 to 110 or more amino acids primarily responsible for antigen recognition. The terms variable light chain (V_{L}) and variable heavy chain (V_{H}) refer to these light and heavy chains, respectively. The remainder of each chain defines a constant region that is conserved and exhibits low variability among different antibodies. Each light chain contains one constant region (C_{L}) and each heavy chain contains three constant regions (C_{H}1, C_{H}2, C_{H}3). Different classes of constant regions in the stem of the antibody generate different isotypes with differing properties based on their amino acid sequence.

Antibodies exist, e.g., as intact immunoglobulins or as a number of well-characterized fragments produced by digestion with various peptidases, referred to herein as “antibody fragments.” Thus, for example, pepsin digests an antibody below the disulfide linkages in the hinge region to produce F(ab)′₂, a dimer of Fab (fragment, antigen binding) which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see, Fundamental Immunology, Paul ed., 3d ed., 1993). While various antibody fragments are defined in terms of the digestion of an intact antibody, one of skill in the art will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

For preparation of antibodies, e.g., recombinant, monoclonal, or polyclonal antibodies, any technique known in the art can be used (see, e.g., Kohler & Milstein, Nature 256:495-497 (1975); Kozbor et al., Immunology Today, 4: 72 (1983); Cole et al., pp. 77-96 in Monoclonal Antibodies and Cancer Therapy, Alan R. Liss, Inc. (1985); Coligan, Current Protocols in Immunology (1991); Harlow & Lane, Antibodies, A Laboratory Manual (1988); and Goding, Monoclonal Antibodies: Principles and Practice (2d ed. 1986)). The genes encoding the heavy and light chains of an antibody of interest can be cloned from a cell, e.g., the genes encoding a monoclonal antibody can be cloned from a hybridoma and used to produce a recombinant monoclonal antibody. Gene libraries encoding heavy and light chains of monoclonal antibodies can also be made from hybridoma or plasma cells. Random combinations of the heavy and light chain gene products generate a large pool of antibodies with different antigenic specificity (see, e.g., Kuby, Immunology (3^{rd} ed. 1997)). Techniques for the production of single chain antibodies or recombinant antibodies (see, e.g., U.S. Pat. Nos. 4,816,567 and 4,946,778) can be adapted to produce antibodies directed to the oligosaccharides of the present invention. In addition, transgenic mice or other organisms such as other mammals may be used to express humanized or human antibodies (see, e.g., U.S. Pat. Nos. 5,545,807; 5,545,806; 5,569,825; 5,625,126; 5,633,425; 5,661,016; Marks et al., Biotechnology, 10:779-783 (1992); Lonberg et al., Nature, 368:856-859 (1994); Morrison, Nature, 368:812-13 (1994); Fishwild et al., Nature Biotechnology, 14:845-51 (1996); Neuberger, Nature Biotechnology, 14:826 (1996); and Lonberg & Huszar, Intern. Rev. Immunol., 13:65-93 (1995)). Alternatively, phage display technology can be used to identify antibodies and heteromeric Fab fragments that specifically bind to selected antigens (see, e.g., McCafferty et al., Nature, 348:552-554 (1990); Marks et al., Biotechnology, 10:779-783 (1992)). Antibodies can also be made bispecific, i.e., able to recognize two different antigens (see, e.g., WO 93/08829; Traunecker et al., EMBO J., 10:3655-3659 (1991); and Suresh et al., Methods in Enzymology, 121:210 (1986)). Antibodies can also be heteroconjugates, e.g., two covalently joined antibodies, or immunotoxins (see, e.g., U.S. Pat. No. 4,676,980; PCT Publications WO 91/00360 and WO 92/200373; and EP 03089).

A “chimeric antibody” is an antibody molecule in which (a) the constant region, or a portion thereof, is altered, replaced, or exchanged so that the antigen binding site (variable region) is linked to a constant region of a different or altered class, effector function, and/or species, or an entirely different molecule which confers new properties to the chimeric antibody, e.g., an enzyme, toxin, hormone, growth factor, drug, etc.; or (b) the variable region, or a portion thereof, is altered, replaced, or exchanged with a variable region having a different or altered antigen specificity. For example, a chimeric antibody can comprise mouse protein sequence in the variable region and human protein sequence in the constant region. A “humanized antibody” comprises even fewer mouse protein sequence in the variable region than chimeric antibodies. Such mouse protein sequence has been replaced by human protein sequence.

In certain instances, the antibody further comprises a detectable label attached thereto. Examples of detectable labels include, without limitation, fluorescein, rhodamine, Texas Red, Cy2, Cy3, Cy5, biotin, horseradish peroxidase, and alkaline phosphatase. One skilled in the art will know of suitable methods for conjugating a particular detectable label to the antibody.

In a further embodiment, the present invention provides a method for treating an inflammatory or infectious disease in an individual in need thereof, the method comprising:
administering to the individual a therapeutically effective amount of an antibody that binds specifically to a unique oligosaccharide.

In certain instances, a therapeutically effective amount of an antibody can be the amount that is capable of preventing or relieving one or more symptoms associated with an inflammatory or infectious disease. Preferably, the antibody binds to a unique oligosaccharide species that is specific to ocular rosacea.

In another aspect, the present invention provides a method for diagnosing an inflammatory or infectious disease in an individual, the method comprising:
detecting the presence or absence of a unique oligosaccharide in a sample from the individual, wherein the presence of the unique oligosaccharide indicates that the individual has the inflammatory or infectious disease.

In one embodiment, the unique oligosaccharide is selected from the group consisting of an O-linked oligosaccharide, an N-linked oligosaccharide, and combinations thereof. In certain instances, detecting the unique oligosaccharide comprises: (a) selectively releasing the oligosaccharides from the sample; (b) obtaining a mass spectrum of the oligosaccharides from the sample using MALDI-FTMS; and (c) determining the presence of the unique oligosaccharide in the mass spectrum. In certain other instances, detecting the unique oligosaccharide comprises contacting the sample with an antibody that binds specifically to the unique oligosaccharide.

In yet another aspect, the present invention provides a method for diagnosing ocular rosacea in an individual, the method comprising:
(a) obtaining a mass spectrum of the oligosaccharides from a sample from the individual using MALDI-FTMS, wherein the oligosaccharides have been selectively released from the sample; and
(b) comparing the mass spectrum from the sample to the mass spectrum from a control sample,
   wherein the presence of a higher abundance of anionic oligosaccharides in the sample indicates that the individual has ocular rosacea.

In one embodiment, the sample from the individual is tear fluid. In another embodiment, the control sample is a sample from an individual not having ocular rosacea. Preferably, the control sample is tear fluid.

In yet another embodiment, the anionic oligosaccharides that are present at a higher abundance in the test sample include anionic O-linked oligosaccharides, anionic N-linked oligosaccharides, and combinations thereof. Examples of specific anionic O-linked oligosaccharides include, without limitation, NeuAc-containing oligosaccharides, NeuGc-containing oligosaccharides, sulfated oligosaccharides, HexA-containing oligosaccharides, and combinations thereof.

In a related aspect, the present invention provides a method for diagnosing ocular rosacea in an individual, the method comprising:
(a) obtaining a mass spectrum of the oligosaccharides from a sample from the individual using MALDI-FTMS, wherein the oligosaccharides have been selectively released from the sample; and
(b) comparing the sum of the absolute intensities of anionic oligosaccharides in the mass spectrum from the sample to the sum of the absolute intensities of anionic oligosaccharides in the mass spectrum from a control sample,
   wherein the presence of a higher value for the sum of the absolute intensities of anionic oligosaccharides in the sample indicates that the individual has ocular rosacea.

Calculating the sum of the absolute intensities of the peaks in a mass spectrum generally provides information about the amount of oligosaccharides present in a sample. For example, the total amount of anionic oligosaccharides present in a sample can be determined by obtaining a mass spectrum of the anionic oligosaccharides and calculating the sum of the absolute intensities of all anionic oligosaccharide species. As such, the term “comparing the sum of the absolute intensities of anionic oligosaccharides in the mass spectrum from the sample to the sum of the absolute intensities of anionic oligosaccharides in the mass spectrum from a control sample” refers to comparing the total amount of anionic oligosaccharides in a test sample to the total amount of anionic oligosaccharides in a control sample and determining any similarity or difference between the two anionic oligosaccharide values. The anionic oligosaccharide value for the control sample can be obtained at the same time as the anionic oligosaccharide value for the test sample or, alternatively, the anionic oligosaccharide value for the control sample can be obtained previously and stored, for example, in a database. One skilled in the art will also appreciate that the two anionic oligosaccharide values can be compared by a computer, e.g., using a computer software program or an internet application.

In one embodiment, the sample from the individual is tear fluid. In another embodiment, the control sample is a sample from an individual not having ocular rosacea. Preferably, the control sample is tear fluid.

In yet another embodiment, the anionic oligosaccharides that are present in the test sample or control sample include anionic O-linked oligosaccharides, anionic N-linked oligosaccharides, and combinations thereof. Examples of specific anionic O-linked oligosaccharides include, but are not limited to, NeuAc-containing oligosaccharides, NeuGc-containing oligosaccharides, sulfated oligosaccharides, HexA-containing oligosaccharides, and combinations thereof.

In still yet another aspect, the present invention provides a method for diagnosing ocular rosacea in an individual, the method comprising:
(a) obtaining a mass spectrum of the oligosaccharides from a sample from the individual using MALDI-FTMS, wherein the oligosaccharides have been selectively released from the sample; and
(b) determining the presence or absence of a marker for ocular rosacea selected from the group consisting of a sulfated oligosaccharide, a NeuAc-containing oligosaccharide, a NeuGc-containing oligosaccharide, a HexA-containing oligosaccharide, a Hex-containing oligosaccharide, and combinations thereof in the mass spectrum,
   wherein the presence of the marker indicates that the individual has ocular rosacea.

As used herein, the term “marker for ocular rosacea” refers to a marker that is indicative of ocular rosacea in an individual. More particularly, the marker for ocular rosacea comprises, for example, a molecule that is specifically present in tear fluid from an individual with ocular rosacea but not present in normal tear fluid; a molecule whose level is increased in tear fluid from an individual with ocular rosacea compared to normal tear fluid; a molecule that is specifically present in normal tear fluid but not present in tear fluid from an individual with ocular rosacea; or a molecule whose level is reduced in tear fluid from an individual with ocular rosacea compared to normal tear fluid. In preferred embodiments of the present invention, the marker for ocular rosacea is an O-linked oligosaccharide. In other embodiments, the marker for ocular rosacea is an N-linked oligosaccharide.

In one embodiment, the sulfated oligosaccharide has a composition selected from the group consisting of [HexNAc]₂[SO₃H], [HexNAc]₂[Hex]₁[SO₃H], [HexNAc]₂[Hex]₂[SO₃H], [HexNAc]₂[Hex]₃[SO₃H], [HexNAc]₂[HexA][SO₃H], [HexNAc]₂[HexA]₁[Hex]₁[SO₃H], [HexNAc]₂[HexA]₁[Hex]₂[SO₃H], and combinations thereof. In another embodiment, the NeuAc-containing oligosaccharide has a composition selected from the group consisting of [HexNAc]₃[NeuAc]₂, [HexNAc]₃[NeuAc]₂[Hex]₁, [HexNAc]₃[NeuAc]₂[Hex]₂, [HexNAc]₃[NeuAc]₂[Hex]₃, and combinations thereof. In yet another embodiment, the NeuGc-containing oligosaccharide has a composition selected from the group consisting of [HexNAc]₁[NeuGc]₃[Hex]₂, [HexNAc]₁[NeuGc]₃[Hex]₃, [HexNAc]₁[NeuGc]₃[Hex]₄, [HexNAc]₁[NeuGc]₃[Hex]₅, and combinations thereof. In still yet another embodiment, the HexA-containing oligosaccharide has a composition selected from the group consisting of m/z 607+[HexA]₁, m/z 607+[HexA]₂, m/z 607+[HexA]₃, m/z 607+[HexA]₄, m/z 607+[HexA]₅, m/z 591+[HexA]₁, m/z 591+[HexA]₂, m/z 591+[HexA]₃, m/z 997+[HexA]₁, m/z 997+[HexA]₂, m/z 575+[HexA]₁, m/z 575+[HexA]₂, m/z 575+[HexA]₃, and combinations thereof. In a further embodiment, the Hex-containing oligosaccharide has a composition selected from the group consisting of m/z 1107+[Hex]₁, m/z 1107+[Hex]₂, m/z 383+[Hex]₁, m/z 383+[Hex]₂, m/z 789+[Hex]₁, m/z 789+[Hex]₂, m/z 1194+[Hex]₁, m/z 1194+[Hex]₂, m/z 1049+[Hex]₁, m/z 1049+[Hex]₂, and combinations thereof.

One skilled in the art will understand that additional methods for diagnosing ocular rosacea in an individual are within the scope of the present invention. In certain instances, a diagnosis of ocular rosacea is made by calculating a ratio of one group of oligosaccharide species to another group of oligosaccharide species, wherein the ratio is indicative of whether the individual has ocular rosacea. As a non-limiting example, the ratio can be calculated by determining the number of NeuGc-containing and NeuAc-containing O-linked oligosaccharide species present in the mass spectrum profile and dividing the number of NeuGc-containing O-linked oligosaccharide species by the number of NeuAc-containing O-linked oligosaccharide species. In certain other instances, a diagnosis of ocular rosacea is made by calculating a percentage of one group of oligosaccharide species, wherein the percentage is indicative of whether the individual has ocular rosacea. As a non-limiting example, the percentage can be calculated by determining the number of sulfated O-linked oligosaccharide species and the total number of O-linked oligosaccharide species present in the mass spectrum profile and dividing the number of sulfated O-linked oligosaccharide species by the total number of O-linked oligosaccharide species. The sulfated/total O-linked oligosaccharide ratio can then be converted into a percentage by multiplying the ratio by 100. Other techniques such as algorithms can be used to calculate the ratios and percentages described above.

In another aspect, the present invention provides an O-linked oligosaccharide having a composition selected from the group consisting of [HexNAc]₂[SO₃H], [HexNAc]₂[Hex]₁[SO₃H], [HexNAc]₂[Hex]₂[SO₃H], [HexNAc]₂[Hex]₃[SO₃H], [HexNAc]₂[HexA][SO₃H], [HexNAc]₂[HexA]₁[Hex]₁[SO₃H], [HexNAc]₂[HexA]₁[Hex]₂[SO₃H], [HexNAc]₃[NeuAc]₂, [HexNAc]₃[NeuAc]₂[Hex]₁, [HexNAc]₃[NeuAc]₂[Hex]₂, [HexNAc]₃[NeuAc]₂[Hex]₃, [HexNAc][NeuGc]₃[Hex]₂, [HexNAc]₁[NeuGc]₃[Hex]₃, [HexNAc]₁[NeuGc]₃[Hex]₄, [HexNAc]₁[NeuGc]₃[Hex]₅, m/z 607+[HexA]₁, m/z 607+[HexA]₂, m/z 607+[HexA]₃, m/z 607+[HexA]₄, m/z 607+[HexA]₅, m/z 591+[HexA]₁, m/z 591+[HexA]₂, m/z 591+[HexA]₃, m/z 997+[HexA]₁, m/z 997+[HexA]₂, m/z 575+[HexA]₁, m/z 575+[HexA]₂, m/z 575+[HexA]₃, m/z 1107+[Hex]₁, m/z 1107+[Hex]₂, m/z 383+[Hex]₁, m/z 383+[Hex]₂, m/z 789+[Hex]₁, m/z 789+[Hex]₂, m/z 1194+[Hex]₁, m/z 1194+[Hex]₂, m/z 1049+[Hex]₁, m/z 1049+[Hex]₂, and combinations thereof. Preferably, the O-linked oligosaccharide is a purified O-linked oligosaccharide.

In one embodiment, the present invention provides an antibody that binds specifically to an O-linked oligosaccharide. In certain instances, the antibody further comprises a detectable label attached thereto. Examples of detectable labels include, without limitation, fluorescein, rhodamine, Texas Red, Cy2, Cy3, Cy5, biotin, horseradish peroxidase, and alkaline phosphatase.

In another embodiment, the present invention provides a method for treating ocular rosacea in an individual in need thereof, the method comprising:
administering to the individual a therapeutically effective amount of an antibody that binds specifically to an O-linked oligosaccharide.

In a preferred embodiment, a therapeutically effective amount of an antibody can be the amount that is capable of preventing or relieving one or more symptoms associated with ocular rosacea.

In yet another embodiment, the present invention provides an oligosaccharide array comprising a plurality of O-linked oligosaccharides immobilized on a solid support. Examples of suitable solid supports include, without limitation, paper, membranes, filters, chips, pins, and glass.

In a further aspect, the present invention provides a kit for diagnosing ocular rosacea in an individual, the kit comprising:
(a) an array comprising a plurality of O-linked oligosaccharides;
(b) a plurality of antibodies that binds specifically to the plurality of O-linked oligosaccharides on the array; and
(c) directions for use of the array and the plurality of antibodies with a sample from the individual.

In certain instances, the plurality of antibodies further comprises a detectable label attached thereto. Examples of detectable labels are described above.

In another aspect, the present invention provides a kit for treating ocular rosacea in an individual in need thereof, the kit comprising:
(a) an antibody that binds specifically to an O-linked oligosaccharide; and
(b) directions for use of the antibody.

### IV. Mass Spectrometry of Oligosaccharides

The complexity of oligosaccharides is readily apparent in the more than 15 million tetrasaccharides with variations in linkages, branching, and anomericity that can be assembled from nine common monosaccharides found in humans. In addition, many common monosaccharides are stereoisomers with identical masses. Several strategies have been developed for the structural elucidation of oligosaccharides that employ nuclear magnetic resonance (NMR) and mass spectrometry. NMR is the only spectroscopic method that provides the complete structure of an oligosaccharide; however, limitations in sensitivity preclude the use of NMR to all but the most abundant species of oligosaccharides (Plancke et al, Eur. J. Biochem., 231:434-439 (1995); Strecker et al., Glycobiology, 5:137-146 (1995); Dell et al., Carbohydr. Res., 115:41-52 (1983)). The analysis is also complex and requires lengthy measurements and interpretation of the NMR spectrum. Furthermore, because oligosaccharide samples are often found to be highly heterogeneous, containing numerous components with abundances that can vary by several orders of magnitudes, rigorous separation is often required for unambiguous elucidation by NMR.

The structural heterogeneity, complexity, and sample limitations make the analyses of oligosaccharides well suited for mass spectrometry (MS). For example, mass spectrometry has been used to identify O-linked oligosaccharides from mucins such as MUC2 (Alving et al., J. Mass Spectrom., 34:395-407 (1999)) and MUC4 (Alving et al., J. Mass Spectrom., 33:1124-1133 (1998)). Fast atom bombardment (FAB)-MS and gas chromatography (GC)-MS have also been used to identify the O-linked oligosaccharides of MUC1 that are associated with breast cancer (Hanisch et al, Eur. J. Biochem., 236:318-327 (1996)). In addition, the N- and O-glycans associated with CA 125 (MUC16) have been characterized by mass spectrometry (Wong et al., J. Biol. Chem., 278:28619-28634 (2003)).

The availability of matrix-assisted laser desorption ionization (MALDI) (Karas et al., Anal. Chem., 60:2299-2301 (1988)) and electrospray ionization (ESI) (Fenn et al., Science, 246:64-71 (1989); Yamashita et al., J. Phys. Chem., 88:4451-4459 (1984)) has significantly increased the sensitivity of mass spectrometry of oligosaccharides (Spengler et al., Anal. Chem., 62:1731-1737 (1990); Stahl et al., Anal Chem., 63:1463-1466 (1991); Powell et al., Rapid Commun. Mass Spectrom., 10:1027-1032 (1996); Harvey et al., Org. Mass Spectrom., 29:753-766 (1994); Cancilla et al., Anal Chem., 70:663-672 (1998)). Collision-induced dissociation (CID) provides considerable structural information. For example, the CID of alkali metal-coordinated species provides information regarding branching and linkage (Dell et al., Carbohydr. Res., 115:41-52 (1983); Aubagnac et al., Org. Mass Spectrom., 18:361-364 (1983); Barofsky et al., Int. J. Mass Spectrom. Ion Phys., 53:319-322 (1983); Burlingame et al., Anal. Chem., 56:417R-467R (1984); Burlingame et al., Anal. Chem., 58:165R-211R (1986); Carroll et al., Anal. Chem., 65:1582-1587 (1993); Dell et al., Mass Spectrom. Rev., 3:357-394 (1984); Domon et al., Glycoconjugate J, 5:397-409 (1988); Angel et al., Carbohydrate Research, 221:17-35 (1991); Dell et al., Int. J. Mass Spectrom. Ion Phys., 46:415-420 (1983); Dell et al., Carbohydr. Res., 120:95-111 (1983); Forsberg et al., J. Biol. Chem., 257:3555-3563 (1982); König et al., J. Am. Soc. Mass Spectrom., 9:1125-1134 (1998)). Stereochemical information is even obtained by coordinating the oligosaccharides to transition metals followed by ESI and CID (Konig et al., supra). However, structure determination using CID requires multiple rounds of tandem MS in order to obtain the complete oligosaccharide sequence. CID of oligosaccharides with pyranose reducing ends also produces the indiscriminate loss of fucose, a common terminating residue, thus masking its position in the chain (Cancilla et al., J. Am. Chem. Soc., 118:6736-6745 (1996); Penn et al., Anal. Chem., 68:2331-2339 (1996)). In addition, the loss of internal saccharide residues through intramolecular rearrangements are encountered with CID (Brüll et al., J. Am. Soc. Mass Spectrom., 8:43-49 (1997)).

The present invention overcomes such limitations by using IRMPD to determine the structure of oligosaccharides. The use of IRMPD is advantageous because the representative fragmentation of oligosaccharides can be obtained down to the last residue. As a result, the complete sequencing of even large oligosaccharides can be performed in a single experiment. Prior to IRMPD analysis, tandem mass spectrometry techniques such as MALDI-Fourier transform mass spectrometry (FTMS) are used to identify oligosaccharide species in a sample (see, Tseng et al., Anal. Biochem., 250:18-28 (1997); Tseng et al., Anal. Chem., 71:3747-3754 (1999)). In particular, the capability of FTMS to provide accurate mass measurements (i.e., <10 ppm routinely, <5 ppm with internal calibration) is critical for obtaining rudimentary oligosaccharide compositions. For example, an oligosaccharide with a quasimolecular ion at m/z 2201.819 has three possible compositions within a tolerance of ±0.1 mass units. Only with a tolerance of 0.01 mass units is the correct composition of two fucoses (Fuc), four hexoses (Hex), and six N-acetylhexosamines (HexNAc) obtained. The use of MALDI-FTMS according to the methods of the present invention can provide the correct composition from a number of possible compositions. The ability to perform multiple stages of tandem MS on ions produced by MALDI is another important feature of the mass spectrometry methods of the present invention.

### V. Examples

The following examples are offered to illustrate, but not to limit, the claimed invention.

### Example 1

### Identification of Oligosaccharide Markers in Tear Fluid Samples from Patients with Ocular Rosacea

This example illustrates the extraction and mass spectrometry analysis of O-linked oligosaccharides from the tear fluid of individuals with ocular rosacea and normal controls.

### Methods

Acquisition of Tear samples. Tears from 37 individuals (21 controls and 16 patients with ocular rosacea) were collected after conjunctival stimulation with a filter paper (Schirmer) strip, using a 10 μl microcapillary tube (Microcaps®; Drummond Scientific Co, Broomall, Pa.). Samples were collected from both eyes (right eye first) as described: 1) the paper strip was placed in the medial third of the inferior lid for 15-30 seconds and removed; 2) with the aid of a slit-lamp, the microcapillary tube was oriented horizontally and its tip was placed to touch the lacrimal meniscus until it was completely filled; and 3) samples from both eyes were immediately transferred to one siliconized low-retention microtube and kept at −80° C. until protein analysis. During collection, the tip of the tube did not touch the skin or eyelashes. The diagnosis of ocular rosacea was based on the standard classification proposed by the National Rosacea Society Expert Committee. All patients presented one or more of the primary features (e.g., flushing, nontransient erythema, papules/pustules, and telangiectasia) with a central face distribution and a history of ocular symptoms compatible with the diagnosis. Patients with epithelial defects, significant conjunctival hyperemia (2+ or more, on a scale of 1-4) and/or corneal ulcers/infiltrates were not included. History of diabetes, contact lens wear, and recent (<1 year) ocular surgery were exclusion criteria for both rosacea patients and controls.

Release of O-linked oligosaccharides from tear fluid by β-elimination. An alkaline sodium borohydride solution (20 μl, mixture of 1.0M sodium borohydride and 0.1M sodium hydroxide) was added to 3 μl of tear fluid. The mixture was incubated at 42° C. for 12 hrs in a water bath. After the incubation, 1.0M hydrochloric acid solution was slowly added in an ice bath to stop the reaction and destroy excess sodium borohydride.

Oligosaccharide purification using a PGC-SPE. O-linked oligosaccharides released by reductive elimination were purified by solid phase extraction using a porous graphitized carbon (PGC) solid phase extraction (SPE) cartridge (Alltech Associates; Deerfield, Ill.). A PGC cartridge was washed with nanopure water followed by 0.05% (v/v) trifluoroacetic acid (TFA) in 80% acetonitrile (ACN) in H₂O (v/v). The solution of released oligosaccharides was applied to the PGC cartridge. Subsequently, the cartridge was washed with nanopure water at a flow rate of approximately 1 ml/min to remove salts and buffer. O-linked glycans were eluted with 20% ACN in H₂O and 40% ACN in 0.05% TFA in H₂O. Each fraction was collected and concentrated in vacuo prior to matrix-assisted laser desorption/ionization mass spectrometry (MALDI MS) analysis.

Mass spectrometric analysis. Mass spectra were recorded on an external source HiResMALDI (IonSpec Corporation; Irvine, Calif.) with a 7.0 Tesla magnet. The HiResMALDI was equipped with a pulsed Nd:YAG laser (266 nm). 2,5-dihydroxybenzoic acid (DHB) was used as a matrix (5 mg/100 μl in 50% ACN in H₂O) for the positive and negative modes. The oligosaccharide solution (1 μl) was applied to the MALDI probe followed by the matrix solution (1 μl). The sample was dried under a stream of air prior to mass spectrometric analysis. Calibration was performed externally using standard oligosaccharide samples. Mass intensities were obtained and used directly from the absolute intensity generated by the mass analyzer. The sum of the absolute intensities of the peaks in a mass spectrum provides information about the amount of oligosaccharides present in the sample.

Statistical analysis: The performance of the identified oligosaccharide markers in detecting ocular rosacea was evaluated by descriptive statistics and receiver operating characteristics curve. The confidence interval for the sensitivity and specificity were calculated by the exact method. All of the statistical analyses were performed using R and STATA 8.0.

### Results

A total of 37 tear fluid samples were analyzed by mass spectrometry. This included samples from 21 individuals without the disease (control samples) and 16 individuals with characteristic ocular rosacea (test samples). Each sample originated from 3 μl of tears. The entire amount was used for subsequent analysis.

The samples were partitioned into two based on the percentage of acetonitrile in water (e.g., 20% or 40%) used to elute the mixture from the PGC cartridge. Smaller oligosaccharides were obtained at the 20% fraction while larger species were obtained at the 40% fraction. Mass spectra were obtained in both the positive (cation) and negative (anion) mode. However, the positive mass spectra did not yield as much information, as the signals were not as abundant. The signals were significantly stronger in the negative mode, indicating that the majority of the oligosaccharides were anionic species containing either sialic acids (N-acetylneuraminic acid (NeuAc) or N-glycolylneuraminic acid (NeuGc)), sulfated, or hexuronic acid residues.

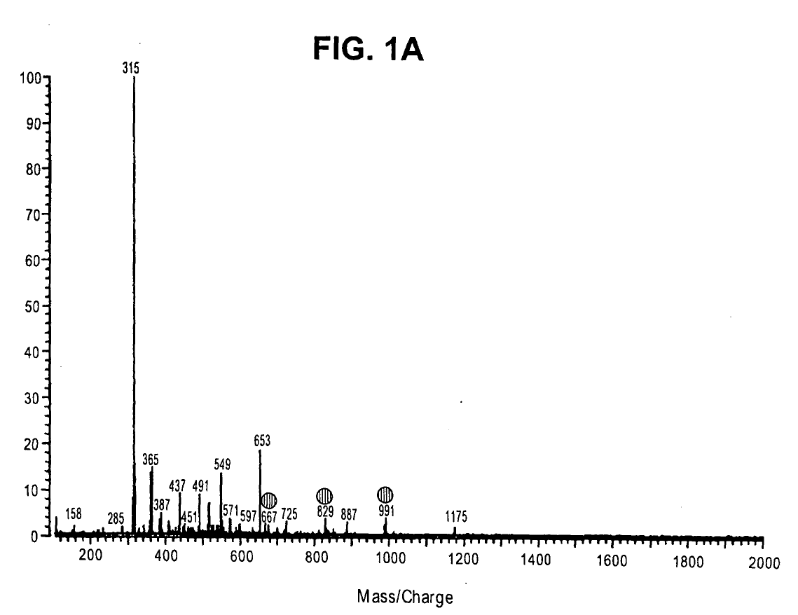

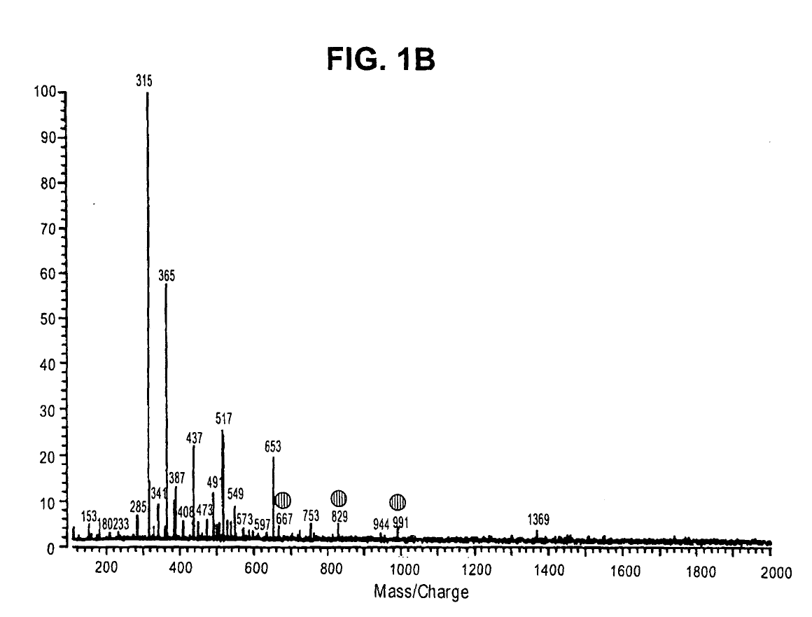

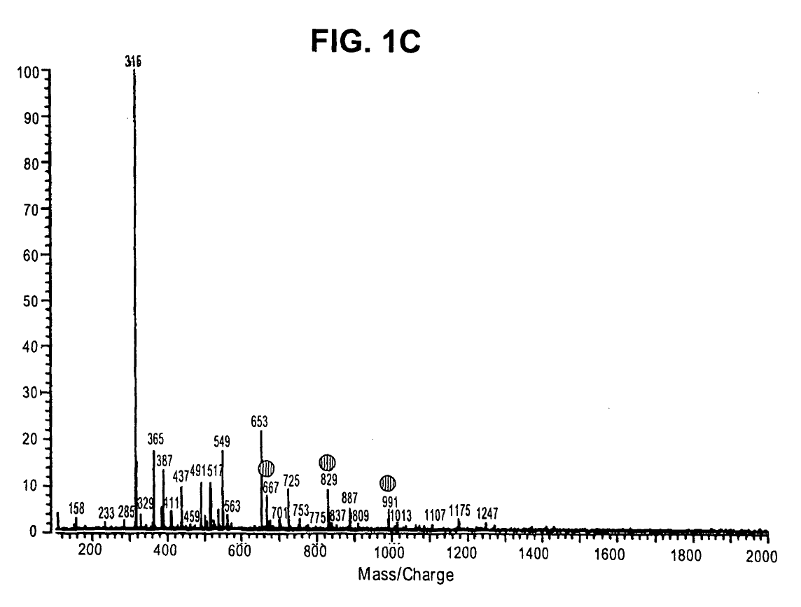

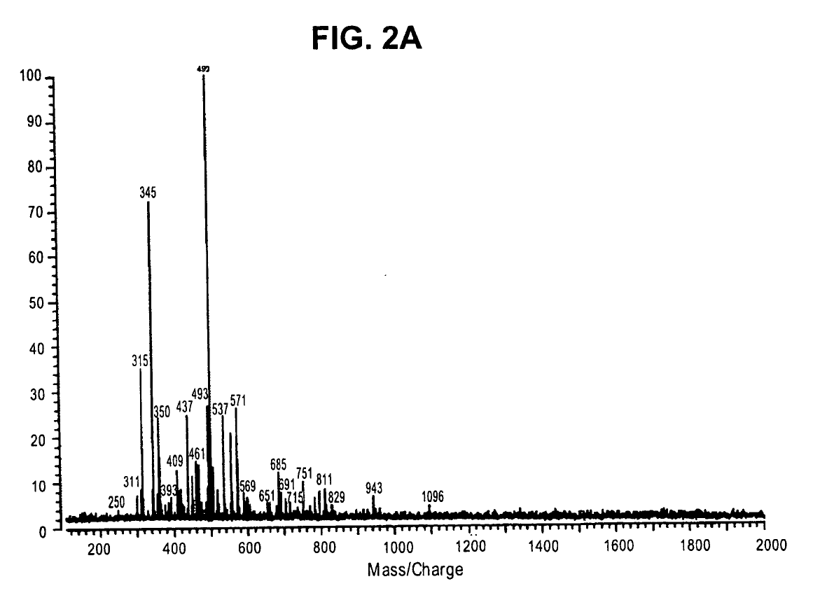

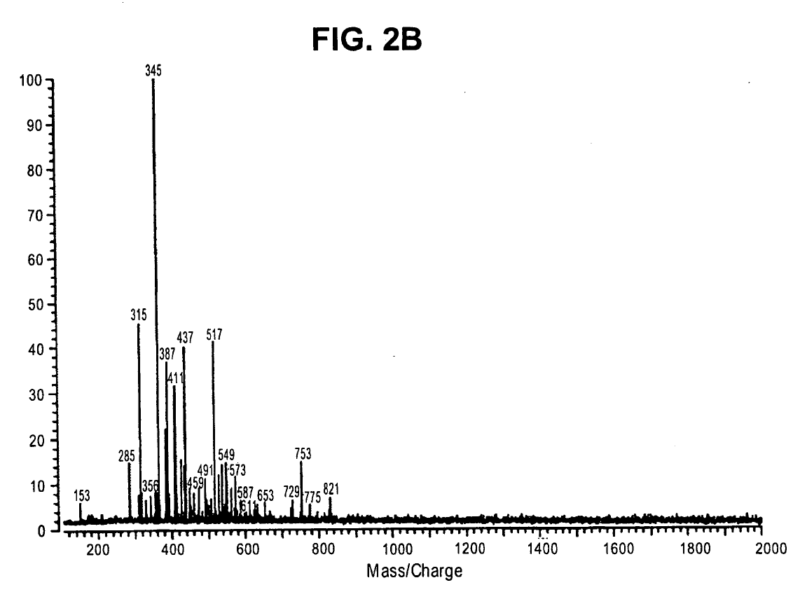

FIGS. 1 and 2 illustrate the mass spectra obtained for control samples using MALDI Fourier transform ion-cyclotron resonance (FT-ICR) mass spectrometry. As shown in Table 1, two common types of spectra were obtained from the 21 control samples tested. In one group composed of seven samples (type A), a series containing an oligomeric sulfated oligosaccharide was obtained having the formula [HexNAc]₂[Hex]ₙ[SO₃H], where HexNAc=N-acetylhexosamine, Hex=hexose, and SO₃H is a sulfate group on either a Hex or HexNAc residue. For these compounds, the number of Hex (n) varied from 0 to 3. These compounds were represented by the ions (theoretical mass in parenthesis) m/z 505.049 (505.133), 667.120 (667.186), 829.169 (829.239), and 991.186 (991.291), which correspond to the deprotonated parent [M-H]⁻. These compounds were found in all three control sample examples shown in FIG. 1. The series does not appear to extend beyond n=3, as n=4 (m/z 1153) was not observed. The remainder of the peaks corresponded either to matrix signal or peptides. A second type of control sample (type B) yielded the mass spectra shown in FIG. 2, which were obtained in the negative mode using MALDI FT-ICR mass spectrometry. These spectra did not appear to contain a significant abundance of oligosaccharide signals; rather, the signals were due to matrix ions and peptides.

TABLE 1
Summary of results obtained from oligosaccharide analyses by MALDI
FT-ICR mass spectrometry of individuals without ocular rosacea.

| | # of | |
|---|---|---|
| Type | Samples | Observations |
| A | 7 | [HexNAc]₂[Hex]ₙ[SO₃H] observed, n = 0-3 |
| B | 10 | No signals corresponding to oligosaccharides observed. |
| | | Signals due to matrix ions and some peptides. |
| C | 4 | One sample (C503) had oligosaccharides similar to |
| | | rosacea patients. Samples C203, C404, and C1304 |
| | | showed oligosaccharides that were not the same as those |
| | | found in rosacea patients. |

Nearly all of the 21 control samples yielded the same spectra as described above except one. This individual (C503) showed peaks in the mass spectrum that were consistent with rosacea. However, it is possible that this individual has either been misdiagnosed or may be suffering from some other ailment.

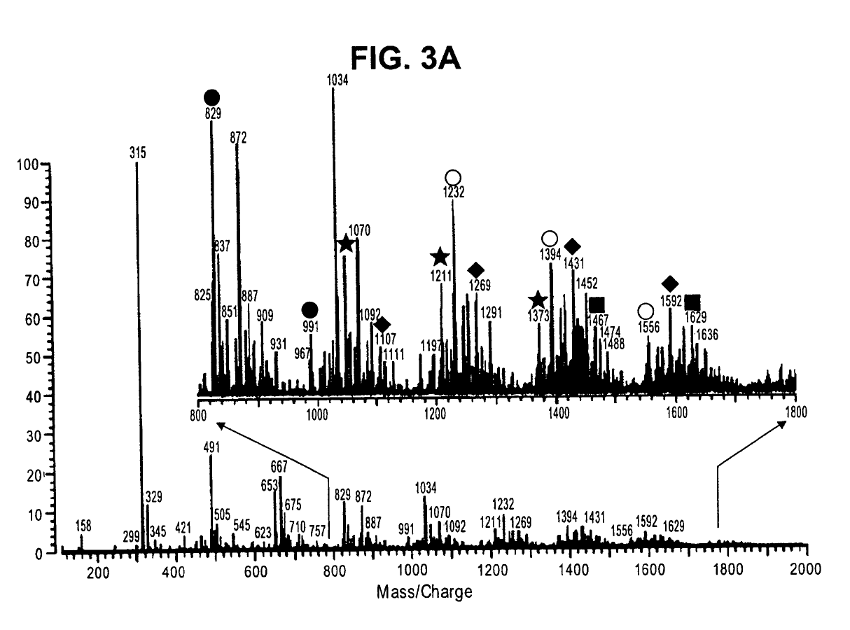

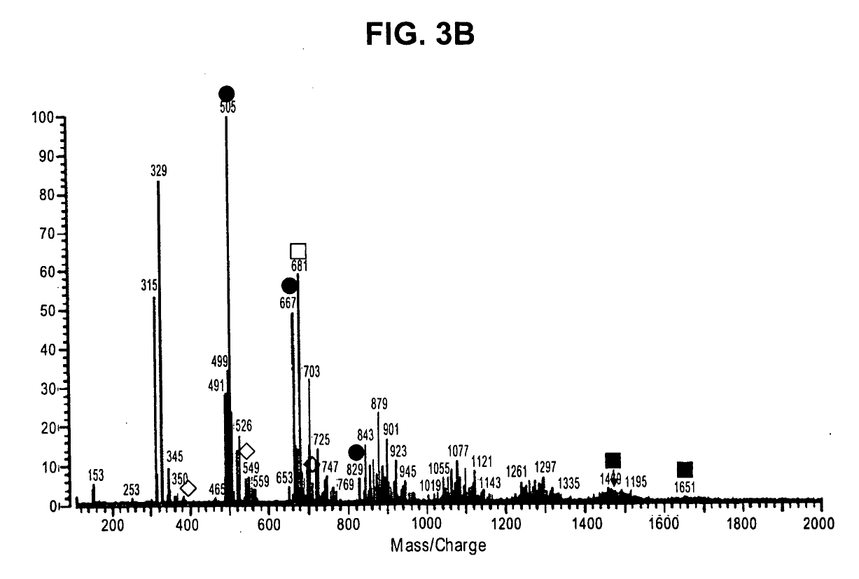

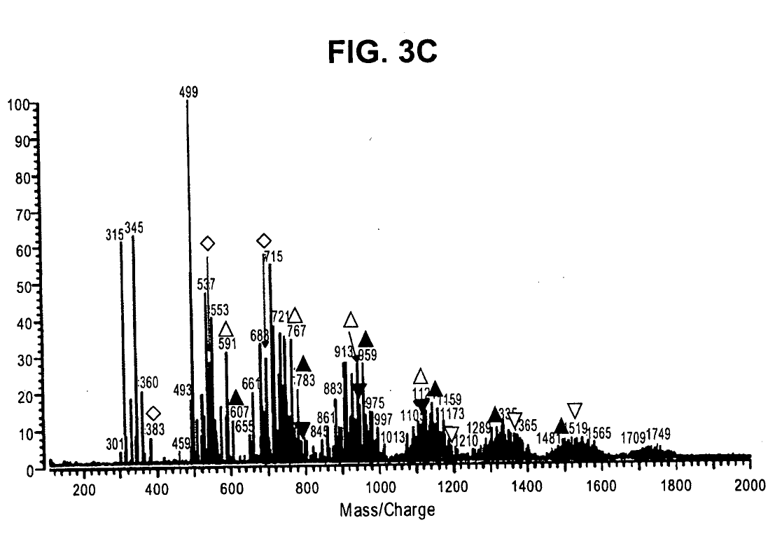

Tear fluid samples from rosacea patients yielded distinctive clusters of peaks that extend to higher masses. To probe the reproducibility of the sample preparation, one patient was analyzed with 3 μl and 30 μl of tear fluid. Both spectra yielded identical results, indicating that the smaller sample size was sufficient for the analysis and that the analysis is highly reproducible. FIG. 3 shows representative spectra for the 20% fractions. The 40% fraction displayed similar characteristics, but with some different masses. The region between m/z 800 and m/z 1800 is expanded in FIG. 3A. A visual comparison between the control and patient samples indicated that nearly all of the control samples did not have masses extending beyond m/z 1000, whereas the patient samples had masses that extended well beyond m/z 1000 and up to as high as m/z 2000. The three test sample examples shown in FIG. 3 illustrate the presence of some heterogeneity among the mass spectra from patients with ocular rosacea. For example, FIG. 3A shows a mass spectrum having predominantly sialylated oligosaccharides (see, also Table 2, Series 2, below). In FIG. 3B, the dominant series corresponded to sulfated oligosaccharides, whereas in FIG. 3C, the dominant series corresponded to hexuronic acid oligosaccharides.

As with some of the control samples, a series with the empirical formula [HexNAc]₂[Hex]ₙ[SO₃H] (Series 1) was also observed in the test samples. Interestingly, this oligosaccharide series was found in 13 of the 16 test samples. The major distinguishing features of the samples from rosacea patients compared to the control samples were the presence of several oligomeric series that were found only in rosacea patients. These series are listed in Table 2. The compounds are the compilation of all oligomeric series found in either 20% fractions, 40% fractions, or both. At least 13 different oligosaccharide series were identified, some with the complete composition determined while others with compositions only partially known. For example, Series 2 has the composition [HexNAc]₃[NeuAc]₂[Hex]ₙ (n=1-3; NeuAc=N-acetylneuranimic acid or sialic acid), with the masses 1231, 1393, 1555, and 1717. The mass difference is equivalent to exactly one hexose group (162.013-experimental, 162.052-theoretical). A series of peaks (Series 3) containing NeuGc is observed having the composition [HexNAc]₁[NeuGc]₃[Hex]ₙ (n=2-5). A second set of sulfated oligosaccharides (Series 4) is observed with the composition [HexNAc]₂[HexA]₁[Hex]ₙ[SO₃H] (n=0-2).

TABLE 2
Summary of the oligosaccharide series the were
found only in individuals with ocular rosacea.

| Series # | Exp. Mass | Theor. Mass | Oligosaccharide composition | Comments |
|---|---|---|---|---|
| 1 () | 505.049 | 505.133 | [HexNAc]₂[SO₃H] | m/z, [M − H]⁻ |
| | 667.120 | 667.186 | [HexNAc]₂[Hex]₁[SO₃H] | |
| | 829.167 | 829.239 | [HexNAc]₂[Hex]₂[SO₃H] | |
| | 991.186 | 991.291 | [HexNAc]₂[Hex]₃[SO₃H] | |
| 2 (◯) | 1230.504 | 1230.415 | [HexNAc]₃[NeuAc]₂ | m/z, [MNa − 2H]⁻ |
| | 1392.511 | 1392.468 | [HexNAc]₃[NeuAc]₂[Hex]₁ | |
| | 1554.531 | 1554.520 | [HexNAc]₃[NeuAc]₂[Hex]₂ | |
| | 1716.578 | 1716.573 | [HexNAc]₃[NeuAc]₂[Hex]₃ | |
| 3 (▪) | 1467.574 | 1467.474 | [HexNAc]₁[NeuGc]₃[Hex]₂ | m/z, [M − H]⁻, |
| | | | | [MNa − 2H]⁻ |
| | | | | 1489.597 |
| | 1629.590 | 1629.527 | [HexNAc]₁[NeuGc]₃[Hex]₃ | m/z, [M − H]⁻, |
| | | | | [MNa − 2H]⁻ |
| | | | | 1651.642 |
| | 1791.615 | 1791.580 | [HexNAc]₁[NeuGc]₃[Hex]₄ | m/z, [M − H]⁻ |
| | 1953.630 | 1953.633 | [HexNAc]₁[NeuGc]₃[Hex]₅ | m/z, [M − H]⁻ |
| 4 (□□) | 681.046 | | [HexNAc]₂[HexA]₁[SO₃H] | m/z, [M − H]⁻, |
| | | | | [MNa − 2H]⁻ |
| | | | | 703.031 |
| | 843.065 | | [HexNAc]₂[HexA]₁[Hex]₁[SO₃H] | m/z, [M − H]⁻, |
| | | | | [MNa − 2H]⁻ |
| | | | | 865.038 |
| | 1005.096 | | [HexNAc]₂[HexA]₁[Hex]₂[SO₃H] | m/z, [M − H]⁻, |
| | | | | [MNa − 2H]⁻ |
| | | | | 1027.070 |
| 5 (▴) | 606.892 | | | m/z? |
| | 782.918 | | 607 + [HexA]₁ | |
| | 958.943 | | 607 + [HexA]₂ | |
| | 1134.997 | | 607 + [HexA]₃ | |
| | 1311.027 | | 607 + [HexA]₄ | |
| | 1487.042 | | 607 + [HexA]₅ | |
| 6 (Δ) | 590.916 | | | |
| | 766.937 | | 591 + [HexA]₁ | |
| | 943.966 | | 591 + [HexA]₂ | |
| | 1119.002 | | 591 + [HexA]₃ | |
| 7 (♦) | 1107.131 | | | |
| | 1269.028 | | 1107 + [Hex]₁ | |
| | 1431.101 | | 1107 + [Hex]₂ | |
| 8 (⋄) | 382.958 | | | |
| | 544.918 | | 383 + [Hex]₁ | |
| | 707.013 | | 383 + [Hex]₂ | |
| 9 (▾) | 788.927 | | | |
| | 950.972 | | 789 + [Hex]₁ | |
| | 1113.010 | | 789 + [Hex]₂ | |
| 10 (∇) | 1193.919 | | | |
| | 1356.016 | | 1194 + [Hex]₁ | |
| | 1518.044 | | 1194 + [Hex]₂ | |
| 11 (★) | 1049.052 | | | |
| | 1211.061 | | 1049 + [Hex]₁ | |
| | 1373.082 | | 1049 + [Hex]₂ | |
| 12 | 996.991 | | | |
| | 1173.090 | | 997 + [HexA]₁ | |
| | 1349.201 | | 997 + [HexA]₂ | |
| 13 | 575.951 | | | |
| | 751.012 | | 576 + [HexA]₁ | |
| | 927.070 | | 576 + [HexA]₂ | |
| | 1103.161 | | 576 + [HexA]₃ | |

A group of anionic oligosaccharides containing hexuronic acid (HexA) residues was also observed. This group included Series 4 (which also contained HexA), 5, 6, 12, and 13. A prominent member of this group is Series 5, characterized by an unknown head group and an increasing number of hexuronic acids, m/z 607+[HexA]ₙ (n=1-5). The mass could contain at least three more HexA residues. Another hexuronic acid series included Series 6, with m/z 591+[HexA]ₙ (n=1-3). The presence of these highly anionic oligosaccharides was supported by the intense signal in the negative mode, corresponding to the deprotonation of the carboxylic acid, and was further supported by the presence of satellite peaks 22 mass units apart, due to carboxylic acids where the acidic hydrogen is replaced by sodium.

Another set of oligosaccharides (i.e., Series 7-11) contained an unknown head group and an elongation of the hexose residue (Hex). Based on the behavior of this signal in the negative spectra, it is likely that the polymeric head group contains an anionic component because the ion signal is strong despite the lack of an anionic group (either a carboxylic acid or a sulfate ester) in the hexose residues.

All 16 test samples showed high degrees of polymerization not observed in the control samples. In particular, Series 1, which was present in 33% of the control samples, was found in 13/16 (81%) of the test samples. As shown in Table 3, there was some diversity in the oligosaccharide content of the test samples. Patients with ocular rosacea did not contain a single set of oligosaccharide markers. However, the tear samples from patients with ocular rosacea were high in hexuronic acid. In fact, HexA oligomers (Series 5, 6, 12, and 13) were found in some form in 12/16 (75%) of the test samples. Series 6, identified in 8/16 (50%) test samples, was the most common HexA oligomer.

TABLE 3
The oligosaccharide series identified in the 16 test samples.

| | Oligosaccharide | Most Abundant |
|---|---|---|
| Sample | Series # | Oligosaccharide |
| R104 | 2, 3, 6, 7, 13 | NeuAc/NeuGc |
| R303 | 1(w), 2, 3, 7, 11 | NeuAc/NeuGc |
| R203 | 1, 4, 7, 8 | sulfated |
| R403 | 1, 3(w), 4, 7(w), 8 | sulfated |
| R304 | 1, 4, 8(w) | sulfated |
| R704 | 1, 4(w), 11, 12 | sulfated |
| R804 | 1, 4, 8(w), 11(w), 13 | sulfated |
| R503 | 1, 4, 6(w), 7(w), 8, 11 | sulfated |
| R504 | 1, 4, 8, 12 | sulfated |
| R404 | 1, 4, 11, 12 | sulfated |
| R103 | 1, 4(w), 5, 6, 8, 13 | HexA |
| R204 | 1, 5, 6, 13 | HexA |
| R904 | 1(w), 5, 6, 13 | HexA |
| R604 | 5, 6, 8, 9, 12, 13 | HexA |
| R1004 | 1(w), 5, 6, 8, 9, 10, 13 | HexA |
| R1104 | 5, 6, 8, 9, 10, 13 | HexA |

The “w” symbolizes weak signals (10%) relative to the other signal in the series.

### Discussion

Oligosaccharide composition of tear and mucins. Based on the compositional analyses of tear samples released by standard O-link procedures, this study shows that human tear fluid from rosacea patients contain a large number of O-linked oligosaccharides. The compositions (number of residues) can be determined in whole or in part based on the accurate mass. Because the method used to release oligosaccharide release primarily O-linked oligosaccharides, they are likely to originate from mucins. The inspection of a number of the components using GlycoSuite® provides important insight into the nature of the oligosaccharides. For example, the species with m/z 667.120 and the composition [HexNAc]₂[Hex][SO₃H] was found to correspond to 10 known structures in mammals, all associated with mucins. In humans, two examples were found with the following structures: HSO₃-6Galβ1-3GlcNAcβ1-3GalNAc and HSO₃-6Galβ1-4GlcNAcβ1-3GalNAc. The second member of Series 2, with m/z 1392.511 and the composition [HexNAc]₃[NeuAc]₂[Hex]₁, yielded 20 structures, all associated with mucins. Nearly all of these are found in humans. The human examples include structures found in colonic and other mucins and in plasma and serum such as, for example, O-linked oligosaccharides attached to the choriogonadotropin beta chain. A member of Series 3, [Hex]₂[HexNAc]₁[NeuGc]₃, is found in the eggs of the Whitespotted Char. However, no NeuGc-containing oligosaccharide has been characterized in humans.

While the accurate mass may provide saccharide compositions, there is no structural information currently available from the mass spectral data alone. Tandem mass spectrometry in the form of collisional-induced dissociation (CID) or infrared multiphoton dissociation (IRMPD) can be useful for providing structural information. However, the size of the oligosaccharides and the comparison with oligosaccharides of similar size all point to the presence of mucins.

Although there have been a number of studies on the proteins, glycoproteins, and mucin glycoproteins in tears, nothing was known about the composition of oligosaccharides in human tears prior to this study. As such, this is the first study to provide a comprehensive characterization of O-linked oligosaccharides that are specific to ocular rosacea.

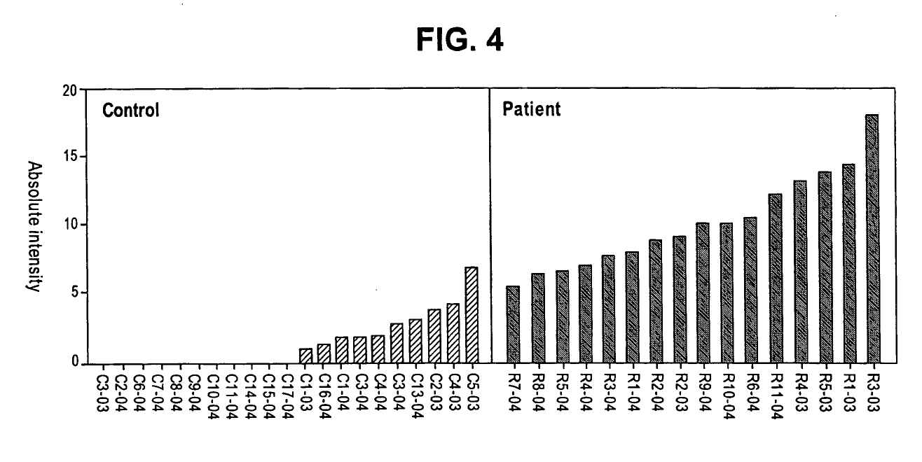

Diagnosing ocular rosacea. This study shows that a simple yet powerful approach for diagnosing ocular rosacea involves determining the presence of a high abundance of oligosaccharides, specifically anionic oligosaccharides, in the tear fluid of an individual. A more precise determination can be performed, for example, based on the presence of hexuronic acid oligomers. As shown in FIG. 4, the sum of the absolute intensities of the anionic oligosaccharides, including sulfated, sialylated, and HexA oligosaccharides, in the control and test samples are arranged in increasing order. Interestingly, the test samples have significantly more anionic oligosaccharides that readily distinguish it from the control samples.

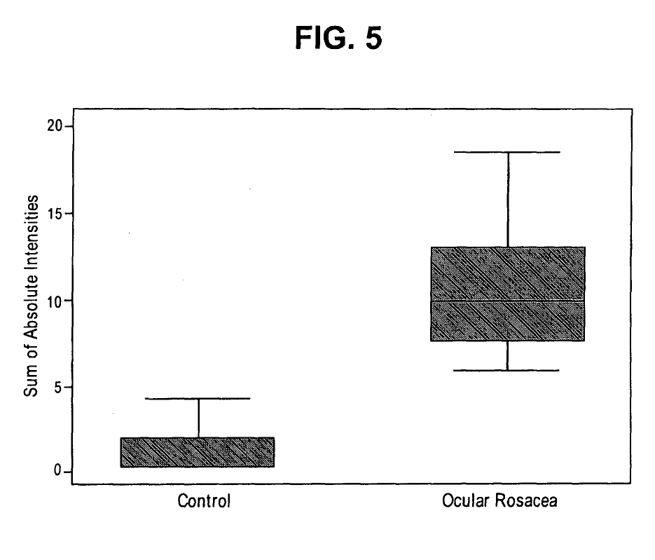

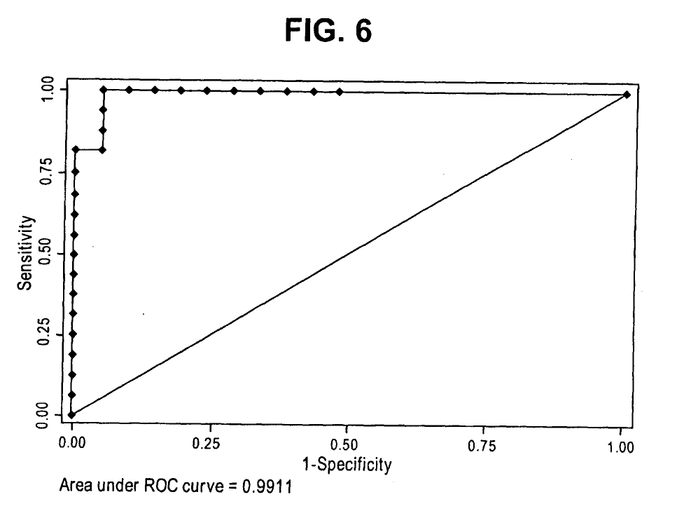

The sum of the absolute intensities of the 13 anionic oligosaccharide series listed in Table 2 can be useful for classifying whether a patient has ocular rosacea. The m/z values for these anionic oligosaccharides have been identified according to their discriminatory pattern in the sample. FIG. 5 shows the distribution of the sum of the absolute intensities as box plots for the control group and the ocular rosacea group. Although there is one outlier in the control group with a relatively larger value compared to the rest of the group, all other values obtained for the control group are less than the minimum values for the ocular rosacea group. This illustrates that a useful marker for identifying ocular rosacea patients can be generated by calculating the sum of the absolute intensities of the 13 anionic oligosaccharide series listed in Table 2. For example, 36 out of 37 individuals were correctly diagnosed using an absolute intensity cut-off value of 5.60, which yielded a sensitivity of 100% (95% CI 79.5-100) and a specificity of 95.2% (95% CI 76.2-99.9). FIG. 6 shows the receiver operator characteristic (ROC) curve of using the sum of the absolute intensities of the anionic oligosaccharide series for diagnosing ocular rosacea. The area under the ROC curve is 0.99 (95% CI 0.97-1.00).

### Example 2

### Oligosaccharide Structure Determination

This example illustrates the determination of rudimentary and fine structures of the O-linked oligosaccharides identified from samples such as tear fluid.

The determination of the O-linked oligosaccharide structures is important for the identification of markers suitable for detecting or diagnosing inflammatory or infectious diseases such as ocular rosacea. As such, the present invention provides a method for O-linked oligosaccharide structure determination using tandem MS to obtain rudimentary structures followed by selective exoglycosidase digestion to verify those structures.

Exoglycosidases, e.g., in the form of glycosidase arrays, are commonly used for the structural elucidation of N-linked oligosaccharides. However, unlike N-linked oligosaccharides, where there is a known number of putative structures, O-linked oligosaccharides have significantly greater combinations of structures. As a result, the use of glycosidase arrays for determining the structures of O-linked oligosaccharides can be both expensive and time-consuming. The present invention overcomes such limitations through the targeted use of exoglycosidase digestion that takes advantage of rudimentary structures obtained from tandem MS experiments (e.g., collision-induced dissociation (CID) or IRMPD). Thus, the procedure for the complete structural elucidation of O-linked oligosaccharides involves the following steps. (1) obtain the exact mass to determine the general composition of the residues (e.g., N-acetylhexose (HexNAc), hexose (Hex), fucose (Fuc), N-acetylneuraminic acid (NeuAc), or N-glycolylneuraminic acid (NeuGc)); (2) determine the rudimentary structure using CID or IRMPD; (3) perform exoglycosidase digestion based upon the rudimentary structure to determine the identity of the residue, the linkage, and the anomeric character of the linkage.

Preferably, O-linked oligosaccharide structures are determined using IRMPD. The use of IRMPD is advantageous because it does not require collision gas, thereby increasing repetition rate and the number of scans per spectra. Further, fragments remain in the degradative beam, often yielding the complete sequence down to the last residue. As a result, the complete sequencing of even large oligosaccharides can be performed in a single tandem MS (MS²) experiment. Standard CID experiments often require to MS³ and even MS⁴ to obtain the last residue in the sequence.

Oligosaccharide structures can be verified using exoglycosidase digestion. For example, an exoglycosidase specific to Gal(β1-4) can be used to determine whether the terminal galactose can be cleaved. Another exoglycosidase, N-acetyl-glucosaminidase, can then be used to determine the position and verify the identity of the GlcNAc residue. IRMPD can also be performed on the exoglycosidase products to verify the positions of the residues.

### Example 3

### Statistical Analyses of Oligosaccharide Markers

This example illustrates the use of statistical analyses to identify oligosaccharide markers while controlling for false positives and to classify and/or predict inflammatory or infectious diseases based upon an oligosaccharide profile.

Statistical classification or prediction analysis of an oligosaccharide profile is similar to statistical analysis of genomic and proteomic data. As such, based upon the oligosaccharide marker profiles obtained from test and control samples, statistical classification/prediction analysis is performed to discriminate between these samples. As a non-limiting example, statistical learning methodologies such as dimension reduction combined with discriminant analysis (Nguyen et al., Bioinformatics, 18:39-50 (2002); Nguyen et al., Bioinformatics, 18:1216-1226 (2002)), support vector machine (Furey et al., Bionformatics, 16:906-914 (2000); Vapnik, Statistical Learning Theory, Wiley-Interscience: New York (1998)), or penalized discriminant analysis (Hastie et al., The elements of statistical learning: Data mining, inference, and prediction, Springer: New York, N.Y. (2001)) can be implemented. Prediction models are constructed based upon a set of training samples. Model validation and error rates for disease prediction based upon an oligosaccharide profile are obtained from an independent data set, consisting of samples not used in the model construction (Ambroise et al., Proc. Natl. Acad. Sci. U.S.A., 99:6562-6566 (2002); Hand, Construction and Assessment of Classification Rules, John Wiley: Chichester, England (1997)).

In addition to using a set of oligosaccharide markers (e.g., approximately 50-70) for disease prediction and classification analyses, statistical analyses can be performed based upon the complete oligosaccharide mass spectrum obtained for each sample. Statistical pattern recognition methods can then be used to identify profiles unique to test samples. For example, dimension reduction techniques such as partial least squares (Nguyen et al., Bioinformatics, 18:39-50 (2002); Nguyen et al., Bioinformatics, 18:1216-1226 (2002)) can be employed due to the large number of molecular species. This technique can also be used to identify a specific oligosaccharide marker or a combination of oligosaccharide markers that are predictive of a particular inflammatory or infectious disease. More particularly, statistical classification and prediction analyses can be used for distinguishing ocular rosacea from other diseases or disorders.

Statistical analysis can also be used to compare the expression levels of oligosaccharides between different groups of samples, such as between test and control samples. For example, statistical methods that have been developed for genomic and proteomic data can be implemented (Efron et al., J. Am. Stat. Assoc., 90:1151-1160 (2001)). Since the number of oligosaccharide markers being compared is relatively large, the false positive discovery rate can be controlled (Nguyen et al., Bioinformatics, 18:39-50 (2002); Nguyen et al., Bioinformatics, 18:1216-1226 (2002); Benjamini et al., J. Royal Stat. Soc. B, 57:289-300 (1995)). Such comparative statistical analyses with false positive discovery rate control can be based upon data obtained from a large number of replications. In addition to sampling replicates, data from technical replicates can be obtained by running multiple experiments using the same sample (i.e., from fixed individual), allowing quantification of the variability of the measurement process. Preferably, technical variability is low relative to sampling variability.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method for identifying an oligosaccharide specific to an inflammatory or infectious disease, said method comprising:
(a) selectively releasing the oligosaccharides from a test sample, wherein said test sample is a sample from an individual having said inflammatory or infectious disease;
(b) obtaining a mass spectrum of the oligosaccharides from said test sample using matrix-assisted laser desorption ionization (MALDI)-Fourier transform mass spectrometry (FTMS); and
(c) comparing the mass spectrum from said test sample to the mass spectrum from a control sample,
wherein said oligosaccharide specific to said inflammatory or infectious disease is identified by the presence of a unique oligosaccharide in the mass spectrum from said test sample.

2. The method of claim 1, wherein said unique oligosaccharide is selected from the group consisting of an O-linked oligosaccharide, an N-linked oligosaccharide, and combinations thereof.

3. The method of claim 1, further comprising the step of subjecting said unique oligosaccharide to infrared multiphoton dissociation (IRMPD).

4. The method of claim 3, further comprising the step of digesting said unique oligosaccharide with an exoglycosidase.

5. The method of claim 1, wherein said control sample is a sample from an individual not having said inflammatory or infectious disease.

6. The method of claim 1, wherein said inflammatory disease is ocular rosacea.

7. The method of claim 6, wherein said test sample is tear fluid.

8. The method of claim 6, wherein said control sample is tear fluid.

9. The method of claim 6, wherein said unique oligosaccharide is an O-linked oligosaccharide selected from the group consisting of an N-acetylneuraminic acid (NeuAc)-containing oligosaccharide, an N-glycolylneuraminic acid (NeuGc)-containing oligosaccharide, a sulfated oligosaccharide, a hexuronic acid (HexA)-containing oligosaccharide, a hexose (Hex)-containing oligosaccharide, and combinations thereof.

10. A method for diagnosing an inflammatory or infectious disease in an individual, said method comprising detecting the presence or absence of a unique oligosaccharide in a sample from said individual, wherein the presence of said unique oligosaccharide indicates that said individual has said inflammatory or infectious disease.

11. The method of claim 10, wherein said unique oligosaccharide is selected from the group consisting of an O-linked oligosaccharide, an N-linked oligosaccharide, and combinations thereof.

12. The method of claim 10, wherein said detecting comprises:
(a) selectively releasing the oligosaccharides from said sample;
(b) obtaining a mass spectrum of the oligosaccharides from said sample using MALDI-FTMS; and
(c) determining the presence of said unique oligosaccharide in the mass spectrum.

13. The method of claim 10, wherein said detecting comprises contacting said sample with an antibody that binds specifically to said unique oligosaccharide.

14. An antibody that binds specifically to a unique oligosaccharide identified by the method of claim 1.

15. The antibody of claim 14, further comprising a detectable label attached thereto.

16. A method for treating an inflammatory or infectious disease in an individual in need thereof, said method comprising:
administering to said individual a composition comprising a therapeutically effective amount of an antibody of claim 14.

17. A method for diagnosing ocular rosacea in an individual, said method comprising:
(a) obtaining a mass spectrum of the oligosaccharides from a sample from said individual using MALDI-FTMS, wherein the oligosaccharides have been selectively released from said sample; and
(b) comparing the mass spectrum from said sample to the mass spectrum from a control sample,
wherein the presence of a higher abundance of anionic oligosaccharides in said sample indicates that said individual has ocular rosacea.

18. The method of claim 17, wherein said control sample is a sample from an individual not having ocular rosacea.

19. The method of claim 17, wherein said anionic oligosaccharides are selected from the group consisting of anionic O-linked oligosaccharides, anionic N-linked oligosaccharides, and combinations thereof.

20. The method of claim 19, wherein said anionic O-linked oligosaccharides are selected from the group consisting of NeuAc-containing oligosaccharides, NeuGc-containing oligosaccharides, sulfated oligosaccharides, HexA-containing oligosaccharides, and combinations thereof.

21. The method of claim 17, wherein said sample is tear fluid.

22. The method of claim 17, wherein said control sample is tear fluid.

23. A method for diagnosing ocular rosacea in an individual, said method comprising:
(a) obtaining a mass spectrum of the oligosaccharides from a sample from said individual using MALDI-FTMS, wherein the oligosaccharides have been selectively released from said sample; and
(b) comparing the sum of the absolute intensities of anionic oligosaccharides in the mass spectrum from said sample to the sum of the absolute intensities of anionic oligosaccharides in the mass spectrum from a control sample,
wherein the presence of a higher value for the sum of the absolute intensities of anionic oligosaccharides in said sample indicates that said individual has ocular rosacea.

24. The method of claim 23, wherein said control sample is a sample from an individual not having ocular rosacea.

25. The method of claim 23, wherein said anionic oligosaccharides are selected from the group consisting of anionic O-linked oligosaccharides, anionic N-linked oligosaccharides, and combinations thereof.

26. The method of claim 25, wherein said anionic O-linked oligosaccharides are selected from the group consisting of NeuAc-containing oligosaccharides, NeuGc-containing oligosaccharides, sulfated oligosaccharides, HexA-containing oligosaccharides, and combinations thereof.

27. The method of claim 23, wherein said sample is tear fluid.

28. The method of claim 23, wherein said control sample is tear fluid.

29. A method for diagnosing ocular rosacea in an individual, said method comprising:
(a) obtaining a mass spectrum of the oligosaccharides from a sample from said individual using MALDI-FTMS, wherein the oligosaccharides have been selectively released from said sample; and
(b) determining the presence or absence of a marker for ocular rosacea selected from the group consisting of a sulfated oligosaccharide, a NeuAc-containing oligosaccharide, a NeuGc-containing oligosaccharide, a HexA-containing oligosaccharide, a Hex-containing oligosaccharide, and combinations thereof in the mass spectrum,
wherein the presence of said marker indicates that said individual has ocular rosacea.

30. The method of claim 29, wherein said sample is tear fluid.

31. The method of claim 29, wherein said sulfated oligosaccharide has a composition selected from the group consisting of [HexNAc]₂[SO₃H], [HexNAc]₂[Hex]₁[SO₃H], [HexNAc]₂[Hex]₂[SO₃H], [HexNAc]₂[Hex]₃[SO₃H], [HexNAc]₂[HexA]₁[SO₃H], [HexNAc]₂[HexA]₁[Hex]₁[SO₃H], [HexNAc]₂[HexA]₁[Hex]₂[SO₃H], and combinations thereof.

32. The method of claim 29, wherein said NeuAc-containing oligosaccharide has a composition selected from the group consisting of [HexNAc]₃[NeuAc]₂, [HexNAc]₃[NeuAc]₂[Hex]₁, [HexNAc]₃[NeuAc]₂[Hex]₂, [HexNAc]₃[NeuAc]₂[Hex]₃, and combinations thereof.

33. The method of claim 29, wherein said NeuGc-containing oligosaccharide has a composition selected from the group consisting of [HexNAc][NeuGc]₃[Hex]₂, [HexNAc]₁[NeuGc]₃[Hex]₃, [HexNAc]₁[NeuGc]₃[Hex]₄, [HexNAc]₁[NeuGc]₃[Hex]₅, and combinations thereof.

34. The method of claim 29, wherein said HexA-containing oligosaccharide has a composition selected from the group consisting of m/z 607+[HexA], m/z 607+[HexA]₂, m/z 607+[HexA]₃, m/z 607+[HexA]₄, m/z 607+[HexA]₅, m/z 591+[HexA]₁, m/z 591+[HexA]₂, m/z 591+[HexA]₃, m/z 997+[HexA]₁, m/z 997+[HexA]₂, m/z 575+[HexA]₁, m/z 575+[HexA]₂, m/z 575+[HexA]₃, and combinations thereof.

35. The method of claim 29, wherein said Hex-containing oligosaccharide has a composition selected from the group consisting of m/z 1107+[Hex]₁, m/z 1107+[Hex]₂, m/z 383+[Hex]₁, m/z 383+[Hex]₂, m/z 789+[Hex]₁, m/z 789+[Hex]₂, m/z 1194+[Hex]₁, m/z 1194+[Hex]₂, m/z 1049+[Hex]₁, m/z 1049+[Hex]₂, and combinations thereof.

36. An O-linked oligosaccharide having a composition selected from the group consisting of [HexNAc]₂[SO₃H], [HexNAc]₂[Hex]₁[SO₃H], [HexNAc]₂[Hex]₂[SO₃H], [HexNAc]₂[Hex]₃[SO₃H], [HexNAc]₂[HexA]₁[SO₃H], [HexNAc]₂[HexA]₁[Hex]₁[SO₃H], [HexNAc]₂[HexA]₁[Hex]₂[SO₃H], [HexNAc]₃[NeuAc]₂, [HexNAc]₃[NeuAc]₂[Hex]₁, [HexNAc]₃[NeuAc]₂[Hex]₂, [HexNAc]₃[NeuAc]₂[Hex]₃, [HexNAc]₁[NeuGc]₃[Hex]₂, [HexNAc]₁[NeuGc]₃[Hex]₃, [HexNAc]₁[NeuGc]₃[Hex]₄, [HexNAc]₁ [NeuGc]₃[Hex]₅, m/z 607+[HexA]₁, m/z 607+[HexA]₂, m/z 607+[HexA]₃, m/z 607+[HexA]₄, m/z 607+[HexA]₅, m/z 591+[HexA]₁, m/z 591+[HexA]₂, m/z 591+[HexA]₃, m/z 997+[HexA]₁, m/z 997+[HexA]₂, m/z 575+[HexA]₁, m/z 575+[HexA]₂, m/z 575+[HexA]₃, m/z 1107+[Hex]₁, m/z 1107+[Hex]₂, m/z 383+[Hex]₁, m/z 383+[Hex]₂, m/z 789+[Hex]₁, m/z 789+[Hex]₂, m/z 1194+[Hex]₁, m/z 1194+[Hex]₂, m/z 1049+[Hex]₁, m/z 1049+[Hex]₂, and combinations thereof.

37. An antibody that binds specifically to an O-linked oligosaccharide of claim 36.

38. The antibody of claim 37, further comprising a detectable label attached thereto.

39. The antibody of claim 38, wherein said detectable label is selected from the group consisting of fluorescein, rhodamine, Texas Red, Cy2, Cy3, Cy5, biotin, horseradish peroxidase, and alkaline phosphatase.

40. A method for treating ocular rosacea in an individual in need thereof, said method comprising:
administering to said individual a composition comprising a therapeutically effective amount of an antibody of claim 37.

41. An oligosaccharide array comprising a plurality of O-linked oligosaccharides of claim 36 immobilized on a solid support.

42. The array of claim 41, wherein said solid support is selected from the group consisting of paper, a membrane, a filter, a chip, a pin, and glass.

43. A kit for diagnosing ocular rosacea in an individual, said kit comprising:
(a) an array of claim 41;
(b) a plurality of antibodies that binds specifically to said plurality of O-linked oligosaccharides on said array; and
(c) directions for use of said array and said plurality of antibodies with a sample from said individual.

44. The kit of claim 43, wherein said plurality of antibodies have a detectable label attached thereto.

45. A kit for treating ocular rosacea in an individual in need thereof, said kit comprising:
(a) an antibody of claim 37; and
(b) directions for use of said antibody.
